(19) United States
(12) Patent Application Publication
(10) Pub. No.: US 2025/0163095 A1
(43) Pub. Date: May 22, 2025

(54) BIS-PROTECTED, ACTIVATED GUANINE MONOMERS

(71) Applicant: Eisai R&D Management Co., Ltd., Bunkyo-ku, Tokyo (JP)

(72) Inventors: Praveen Kumar VEMULA, Arlington, MA (US); Hyeong Wook CHOI, Andover, MA (US); Francis G. FANG, Andover, MA (US); Mingde David SHAN, Andover, MA (US)

(73) Assignee: Eisai R&D Management Co., Ltd., Bunkyo-ku, Tokyo (JP)

(21) Appl. No.: 18/842,196

(22) PCT Filed: Mar. 1, 2023
(86) PCT No.: PCT/US2023/014247

§ 371 Date: Aug. 28, 2024

Related U.S. Application Data
(60) Provisional application No. 63/315,280, filed on Mar. 1, 2022.

(51) Int. Cl. C07H 19/173; C07F 9/6561

(52) U.S. Cl. CPC C07H 19/173; C07F 9/65616

(57) ABSTRACT

Bis-protected, activated guanine monomers or pharmaceutically acceptable salts thereof for use in the synthesis of polymorpholino oligonucleotides and methods of making the bis-protected, activated guanine monomers are provided.

### CROSS-REFERENCE TO RELATED APPLICATIONS

This application claims the benefit of priority of U.S. Provisional Patent Application No. 63/315,280, filed on Mar. 1, 2022. That application is incorporated by reference as if fully rewritten herein.

### FIELD

The present disclosure relates to bis-protected, activated guanine monomers, methods of their synthesis and their use in the production of antisense oligonucleotides.

### BACKGROUND

Antisense oligonucleotides (ASO) are used in the modulation of gene expression in a sequence-specific manner. They have been developed for target validation and therapeutic purposes. Antisense technology has the potential to cure diseases caused by the expression of harmful genes, including diseases caused by viral infections, cancer growth, neuronal degradation (i.e., Alzheimer's) and inflammatory diseases. Optimized antisense oligonucleotides (ASOs) can be used to target primary gene transcripts, mRNA product(s), spliced and unspliced coding and noncoding RNAs.

ASOs modulate RNA function by two broad mechanisms. A steric blocking mechanism that could lead to splicing modulation, non-sense mediated decay (NMD) and translation blocking. And RNase H-mediated degradation that results in cleavage of the target RNA by making an RNA-ASO heteroduplex.

### BRIEF SUMMARY

Phosphorodiamidate morpholino oligomers (PMO) are short single-stranded DNA analogs that contain a backbone of morpholine rings connected by phosphorodiamidate linkages. They have been reported to be useful in certain therapies.

One nucleic acid monomer that is modified for the production of PMOs is guanine. The guanine base of these monomers can be modified to protect the guanine base from participating in side reactions during the production of PMOs. When the guanine base of guanine monomers is mono-protected, stability issues begin to arise. In light of this feature of the guanine monomers, we provide bis-protected, activated guanine monomers with improved stability relative to mono-protected guanine monomers. These more stabilized guanine monomers can be used to produce PMOs with a lower incidence of by-product formation and in higher yields.

One embodiment is a bis-protected, activated guanine monomer or a pharmaceutically acceptable salt thereof. A monomer is said to be “activated” when that monomer has been prepared for use in further steps leading to synthesis of a dimer or oligomer.

In some embodiments, the bis-protected, activated guanine monomer includes an activated morpholine ring according to Formula I

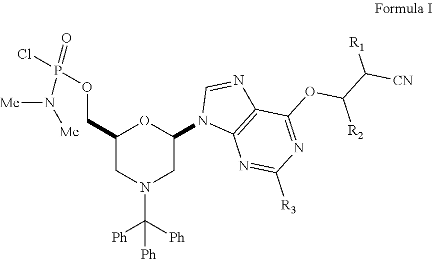

wherein R₁, R₂ is selected from H, (R)-methyl or (S)-methyl, C₁-C₄ alkyl, phenyl, aryl, cycloalkyl or any combination thereof; and
wherein R₃ is selected from NH₂, —NHC(O)R₇, —NHC(O)OR₇,

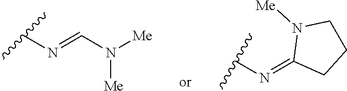 [SMILES: */N=C/N(C)C ; */N=C1/CCCN1C]

and where R₇ may be a C₁-C₆ alkyl, isopropyl, 2,2,2-trichloroethyl, benzyl or aryl.

In some embodiments, R₁ and R₂ can be linked together to form a C₃ to C₇ cycloalkyl ring or a heterocycle ring comprising oxygen and/or nitrogen, all of which may be saturated or unsaturated, and may be substituted at one or more carbon atoms with a C₁-C₆ alkyl.

In other embodiments, the bis-protected, activated guanine monomer is a stereoisomer of Formula I. Without being limited to the following structures, some embodiments of the bis-protected, activated guanine monomer comprise a stereoisomeric structure according to one of Formula (Ia) and (Ib).

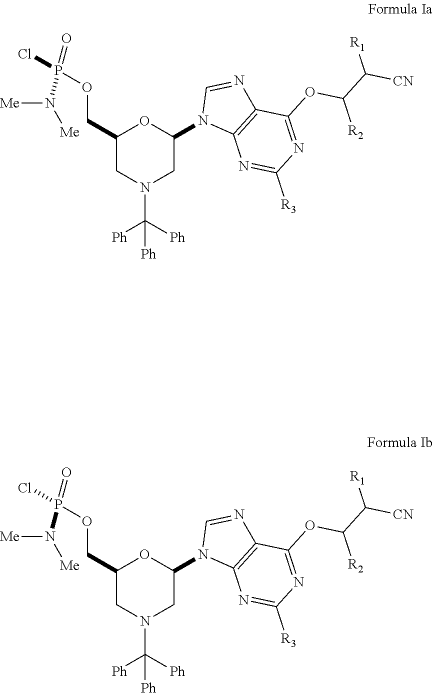

In other embodiments, the bis-protected, activated guanine monomer comprises an activated tetrahydrofuran ring according to Formula II:

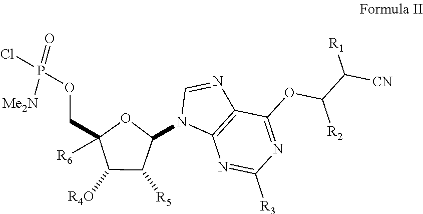

wherein R₁, R₂ is selected from a H, (R)-methyl or (S)-methyl, C₁-C₄ alkyl, phenyl, aryl, cycloalkyl or any combination thereof;
wherein R₃ is selected from NH₂, —NHC(O)R₇, —NHC(O)OR₇,

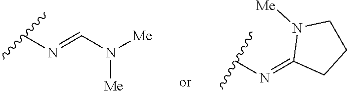 [SMILES: */N=C/N(C)C ; */N=C1/CCCN1C]

and where R₇ may be a C₁-C₆ alkyl, isopropyl, 2,2,2-trichloroethyl, benzyl or aryl;
wherein R₄ is selected from H, trityl (Tr), monomethoxytrityl (MMTr), dimethoxytrityl (DMTr), —Si(R₈)₃, where R₈ is C₁-C₆ alkyl or aryl; and
wherein R₅ is selected from H, —OMe, —F or —OCH₂CH₂OMe.

In other embodiments, R₅ and R₆ can be linked together to form a C₃ to C₇ cycloalkyl ring or heterocycle ring comprising oxygen and/or nitrogen, all of which may be saturated or unsaturated, and may be unsubstituted or substituted with C₁-C₆ alkyl. For example, in one embodiment, R₅ and R₆ can be linked together to form S-cEt, as depicted (in the context of the overall molecule) below:

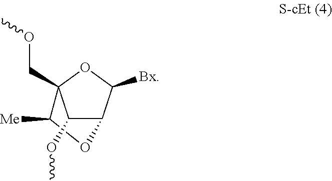

In another embodiment R5 and R6 form LNA, which has the structure depicted (in the context of the overall molecule) below:

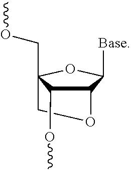

In other embodiments, R₁ and R₂ can be linked together to form a C₃ to C₇ cycloalkyl ring or heterocycle ring comprising oxygen and/or nitrogen, all of which may be saturated or unsaturated, and may be unsubstituted or substituted with C₁-C₆ alkyl.

In some embodiments, the bis-protected, activated guanine monomer is a stereoisomer of Formula II. Without being limited to the following structures, some embodiments of the bis-protected, activated guanine monomer comprise a stereoisomeric structure according to Formulas (IIa) and (IIb).

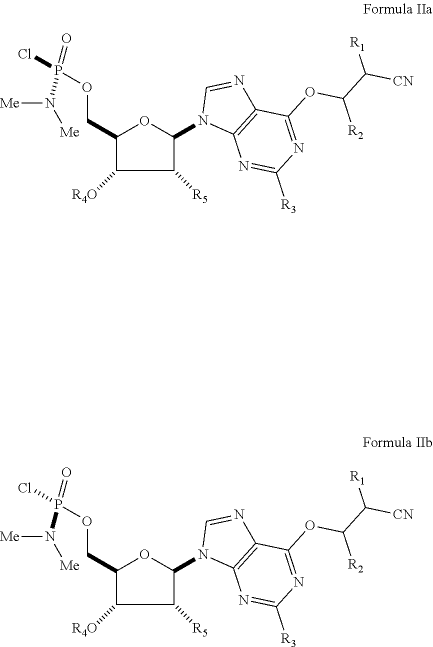

In other embodiments, the bis-protected, activated guanine monomer could be represented by the following structures:

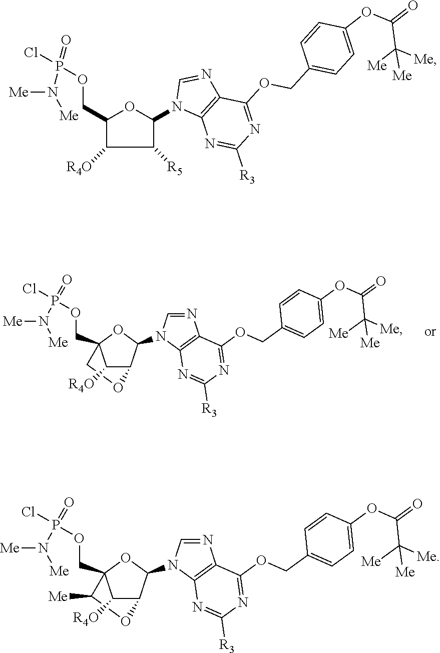

In some embodiments, the bis-protected, activated guanine monomer possesses the following structure:

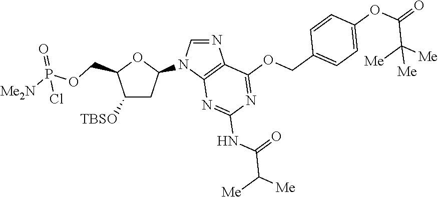 [SMILES: CC(C)C(=O)Nc1nc(OCc2ccc(OC(=O)C(C)(C)C)cc2)c2ncn([C@H]3C[C@H](O[Si](C)(C)C(C)(C)C)[C@@H](COP(=O)(Cl)N(C)C)O3)c2n1]

The bis-protected, activated guanine monomers described herein may be produced from a process comprising:
i) reacting a protected guanine monomer according to Formula (III):

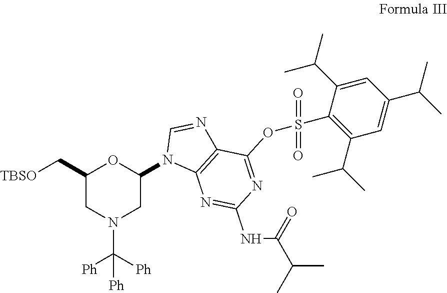 [SMILES: CC(C)C(=O)Nc1nc(OS(=O)(=O)c2c(C(C)C)cc(C(C)C)cc2C(C)C)c2ncn([C@H]3CN(C(c4ccccc4)(c4ccccc4)c4ccccc4)C[C@@H](CO[Si](C)(C)C(C)(C)C)O3)c2n1]

with an alcohol in the presence of a base to produce a protected guanine intermediate according to Formula IV:

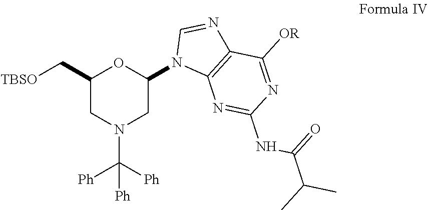

wherein R is

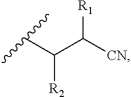

wherein R₁ and R₂ are selected from H, (R)-methyl or (S)-methyl, C₁-C₄ alkyl, phenyl, aryl, cycloalkyl or any combination thereof, including wherein R may be a group selected from the following structures:

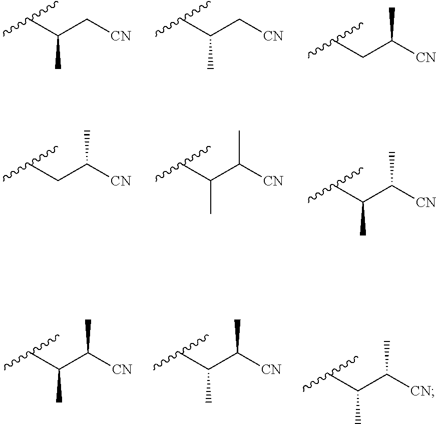

ii) reacting the protected guanine intermediate according to Formula IV with triethylamine trihydrofluoride to produce a deprotected guanine intermediate according to Formula V:

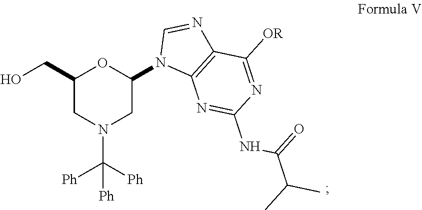

and
iii) reacting the deprotected guanine intermediate according to Formula V with lithium bromide, a second activating agent and N,N-dimethylphosphoramic dichloride to create the bis-protected, activated guanine monomers described herein.

In some embodiments, the reagent used in step (i) is N-methylpyrrolidine, 1,4-diazabicyclo[2.2.2]octane (DABCO), quinuclidine, trimethylamine and any combination thereof.

In other embodiments, the base used in step (i) is 1,8-Diazabicyclo[5.4.0]undec-7-ene (DBU), diisopropyl ethylamine, potassium carbonate, potassium tert-butoxide, sodium hydride, Na₂CO₃, CsCO₃, pyrrolidine, triethylamine, pyridineand any combination thereof.

In some embodiments, the alcohol used in step (i) may be 3-hydroxy-2-methylpropanenitrile, 3-hydroxy-3-methylpropanenitrile or 2,3-dimethyl-3-hydroxymethylpropanenitrile. The alcohol used in step (i) may also be any stereoisomer of 3-hydroxy-2-methylpropanenitrile, 3-hydroxy-3-methylpropanenitrile or 2,3-dimethyl-3-hydroxymethylpropanenitrile.

In some embodiments, the second activating agent used in step (iii) is DBU, 2,6-lutidine, N-methyl imidazole, 1H-tetrazole, 4,5-dichloroimidazole, 4,5-dicyanoimidazole, LiHMDS, 4-ethylmorpholine, DMAP, triethylamine, pyridine, Hunig's base and any combination thereof.

In some embodiments, the reaction of step (i), (ii) or (iii) may further comprise a solvent. The solvent may be DCM, ethyl acetate, acetonitrile, THF, toluene, dimethylsulfoxide, dimethylacetamide, DMFor any combination thereof.

In some embodiments, the bis-protected, activated guanine monomers described herein may be produced from a process comprising any, and in some embodiments all, of the following steps:
i) reacting a guanine monomer according to Formula VI:

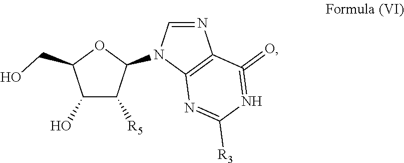

with a first protecting agent to produce a first protected guanine monomer according to Formula (VII):

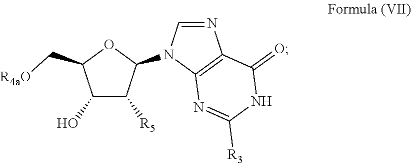

ii) reacting the protected guanine monomer of Formula (VII) with a second protecting agent to produce a protected guanine monomer according to Formula (VIII):

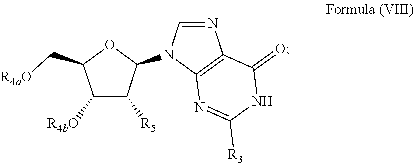

iii) reacting the second protected guanine monomer of Formula (VIII) with an activating agent to produce a protected guanine monomer according to Formula (IX):

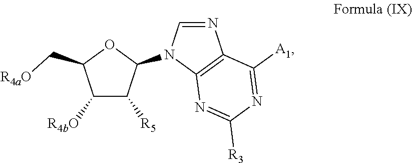

wherein A₁ is a leaving group formed from the reaction with the activating agent;
iv) reacting the protected guanine monomer of Formula (IX) with a selected alcohol to produce a protected guanine monomer according to Formula (X):

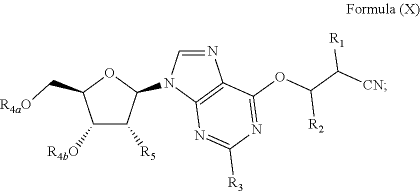

v) deprotecting the protected guanine monomer according to Formula (X) with a deprotecting agent to produce a protected guanine monomer according to Formula (XI):

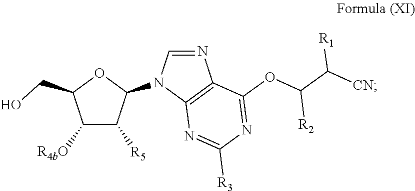

vi) reacting the protected guanine monomer according to Formula (XI) with an electrophile to produce a protected guanine monomer according to Formula II.

In some embodiments, step iv) comprises reacting the protected guanine monomer of Formula (IX) with 4-(hydroxylmethyl)phenyl pivalate to produce a protected guanine monomer according to Formula (XII):

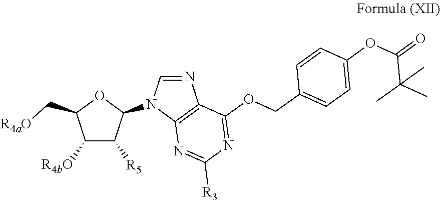

In other embodiments, step v) comprises reacting the protected guanine monomer according to Formula (XII) with a deprotecting agent to produce a protected guanine monomer according to Formula (XIII):

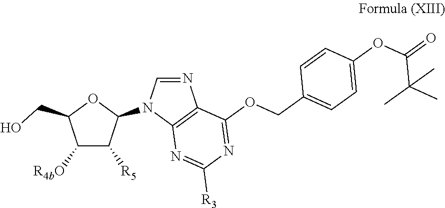

In some embodiments, step vi) comprises reacting the protected guanine monomer according to Formula (XIII) with an electrophile to produce a compound with the following structure:

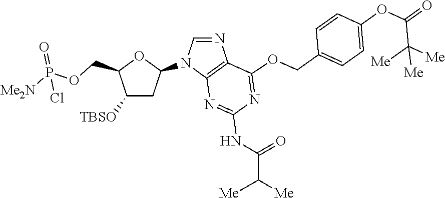 [SMILES: CC(C)C(=O)Nc1nc(OCc2ccc(OC(=O)C(C)(C)C)cc2)c2ncn([C@H]3C[C@H](O[Si](C)(C)C(C)(C)C)[C@@H](COP(=O)(Cl)N(C)C)O3)c2n1]

In some embodiments, the first protecting agent can be trityl chloride, 4-monomethoxytrityl chloride, 4,4′-Dimethoxytrityl chloride, or a silyl chloride comprising the formula (Si(R₆)₃)Cl, wherein R₆ is C₁-C₆ alkyl or aryl. In some embodiments, the silyl chloride is tert-Butyldimethylsilyl chloride.

In other embodiments, the second protecting agent can be trityl chloride, 4-monomethoxytrityl chloride, 4,4′-Dimethoxytrityl chloride, or a silyl chloride comprising the formula (Si(R₆)₃)Cl, wherein R₆ is C₁-C₆ alkyl or aryl. In some embodiments, the silyl chloride is tert-Butyldimethylsilyl chloride.

In other embodiments, the activating agent is 2,4,6-Triisopropylbenzenesulfonyl chloride.

In some embodiments, A₁ is

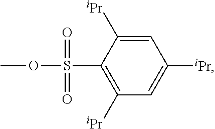 [SMILES: COS(=O)(=O)c1c(C(C)C)cc(C(C)C)cc1C(C)C]

arylsulfonyl which may be substituted with 1 to 3 alkyl groups, trifluromethanesulfonyl, methylsulfonyl and the combination thereof.

In other embodiments, the alcohol in step iv) is

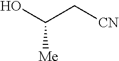

or 4-(hydroxymethyl)phenyl pivalate.

In other embodiments, the electrophile is

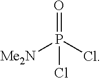

Additional reagents and/or solvents can be added to any one or more of steps i) to vi). These reagents can be selected from the either DBU, DMAP, triethylamine, N-methylpyrrolidine, LiBr, 2,6-Lutidine, N-methylimidazole or combinations thereof. The solvents added to any one or more of steps i) to vi) can be either DCM, THF, MeCN, toluene, DMF, water or combinations thereof.

### BRIEF DESCRIPTION OF THE DRAWINGS

FIG. 1A and FIG. 1B illustrate possible structures of the bis-protected, activated guanine monomers described herein.

FIG. 2 depicts a possible side-reaction that can occur during PMO synthesis with a conventional cyanoethyl protection on a guanine base.

FIG. 3A-3D depict the reactivities of a PMO thymine monomer with acrylonitrile, β-methyl acrylonitrile and α-methyl acrylonitrile under various conditions.

FIG. 4A and FIG. 4B illustrate the occurrence of cyanoethyl deprotection of modified guanine monomers during an activation step.

FIG. 5A and FIG. 5B depict the chiral separation spectra of bis-protected, activated guanine monomers.

### DETAILED DESCRIPTION

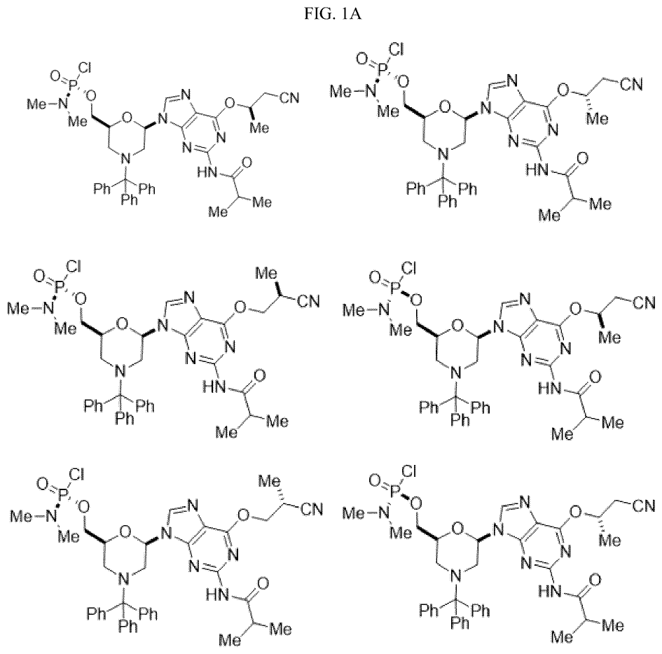

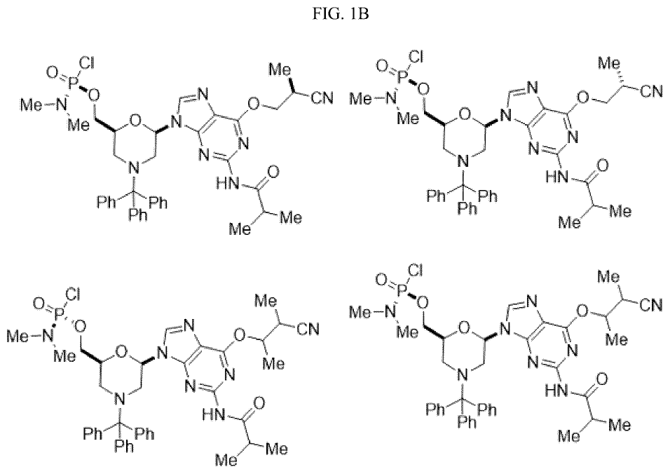

An aspect of the present disclosure is directed to bis-protected, activated guanine monomers. The bis-protected, activated guanine monomers may comprise a morpholine ring and have a structure depicted in FIG. 1A and FIG. 1B or represented by Formula I, Ia or Ib.

The bis-protected, activated guanine monomers may also comprise a tetrahydrofuran ring and have a structure represented by Formula II, IIa or IIb.

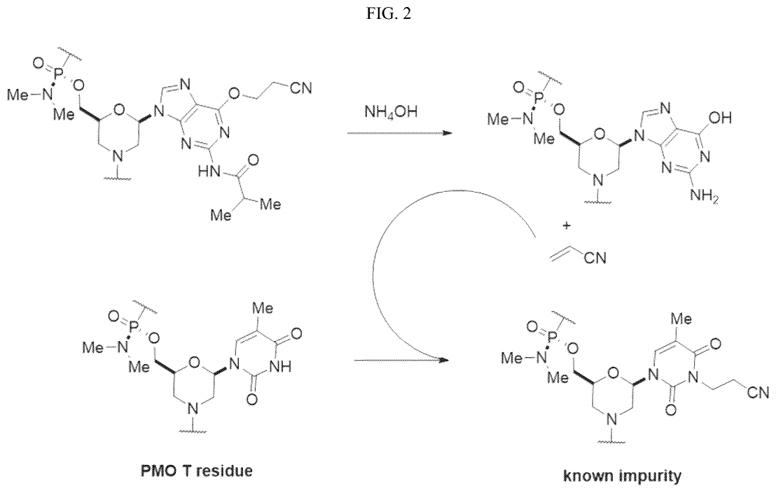

One utility of the bis-protected, activated guanine monomers described herein is improvement of synthesis of PMO by reducing the occurrence of side-reactions between de-protected guanine residues and thymine residues (see FIG. 2).

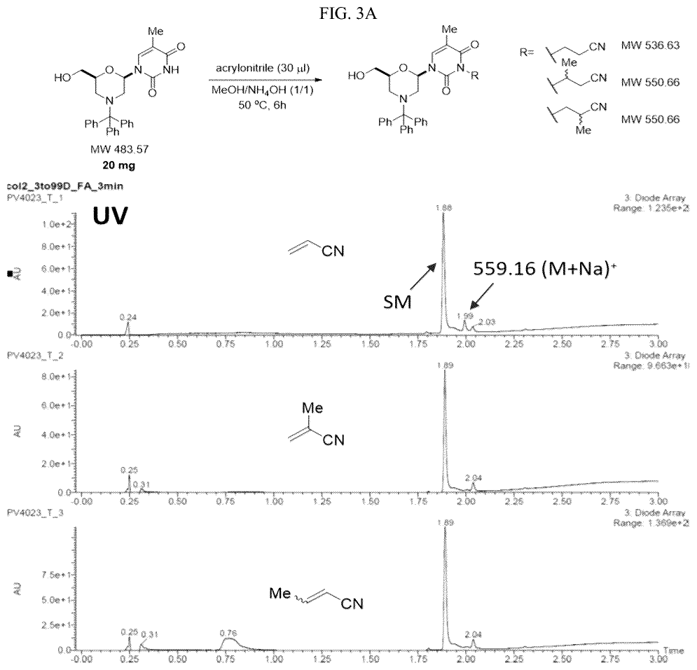

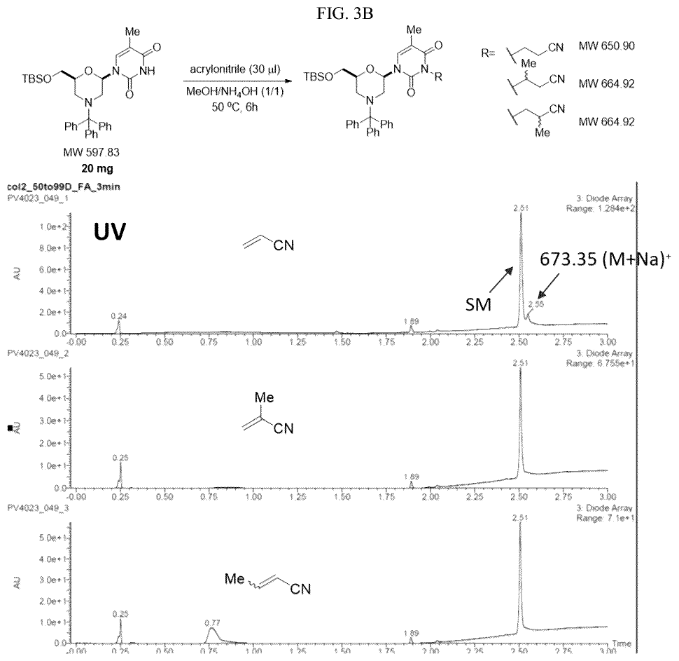

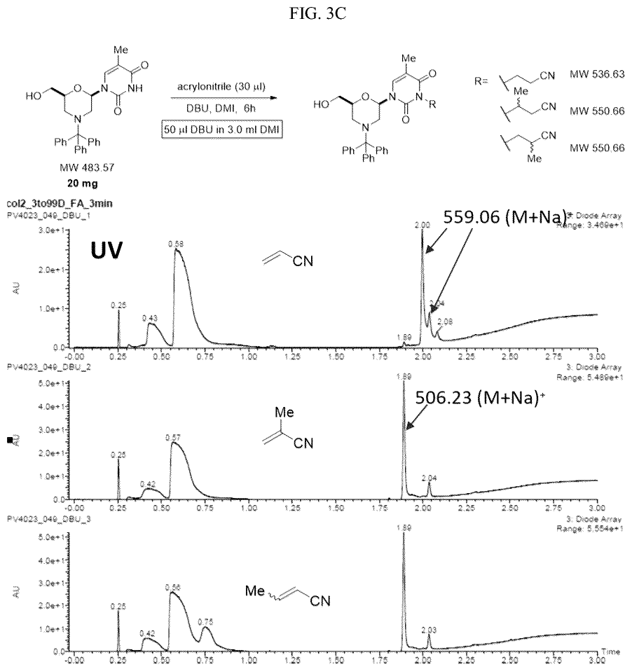

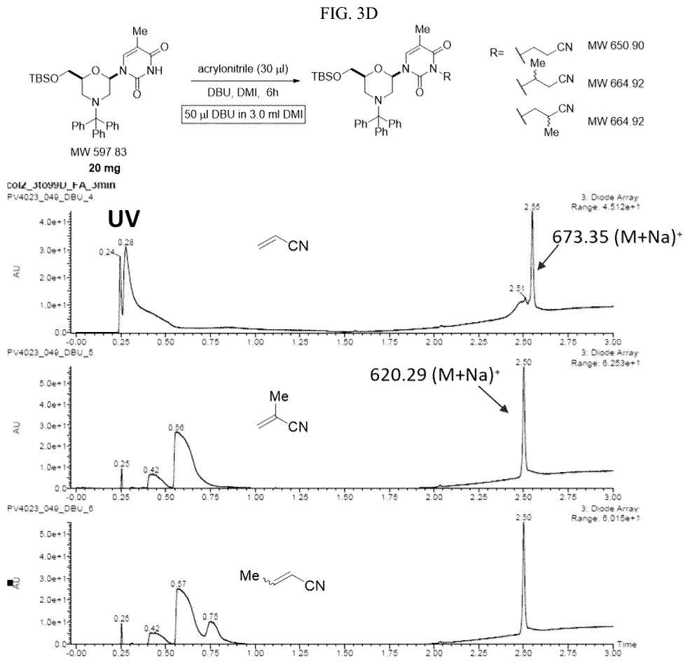

When conventional cyanoethyl protecting groups are installed onto a guanine base, the deprotection of that cyanoethyl group generates acrylonitrile, which can react with a thymine residue in a PMO to give an alkylated impurity (see FIG. 3A-3D). When a β-methyl group or an α-methyl group is installed onto the cyanoethyl protecting group, the deprotection of these groups leads to less reactive by-products (i.e., β-methyl acrylonitrile or α-methyl acrylonitrile), which can lead to improved reaction yields (see FIG. 3A-3D).

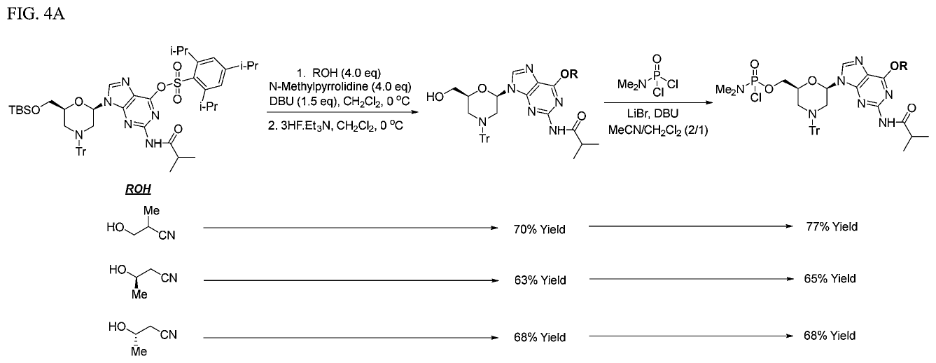

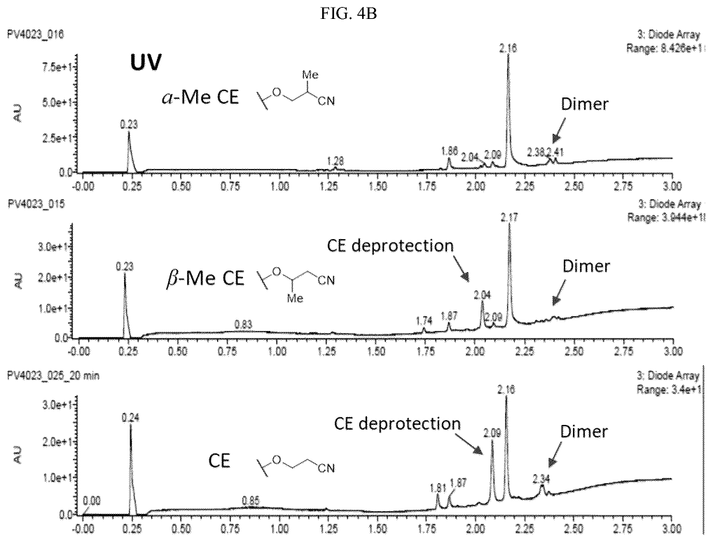

Additionally, use of a β-methylated or an α-methylated cyanoethyl protecting group for guanine monomers has been found to reduce the occurrence in the loss of the cyanoethyl protecting group during activation of the guanine monomers described herein. FIG. 4A depicts a synthesis scheme wherein a cyanoethyl protecting group is first installed onto a guanine monomer and afterwards, the guanine monomer is activated. FIG. 4B shows the occurrence of the deprotection side-reaction that can occur during the activation step by detecting the deprotected species with HPLC. The α-methylated cyanoethyl protecting group produced a more stable guanine monomer when compared to guanine monomers that were protected with conventional cyanoethyl protecting groups during the activation step since the α-methylated cyanoethyl protected guanine monomers produced a cleaner reaction profile and gave the desired product in higher yields (77% vs. 43%). The β-methylated cyanoethyl protected guanine monomers also showed better stability and higher yields when compared to guanine monomers protected with conventional cyanoethyl protection (60% vs. 43%). The bis-protected, activated guanine monomers were also found to reduce the risk of forming acrylate side product during ASO synthesis and were shown to increase chloride stability.

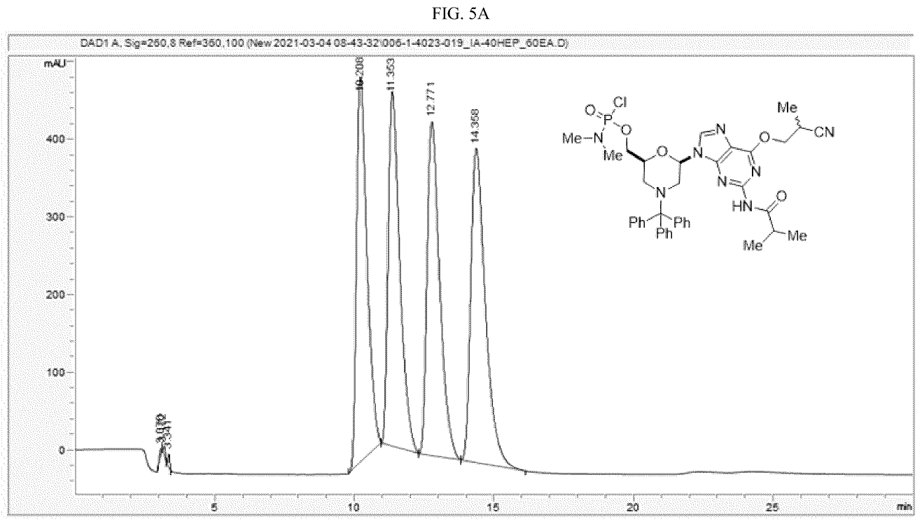

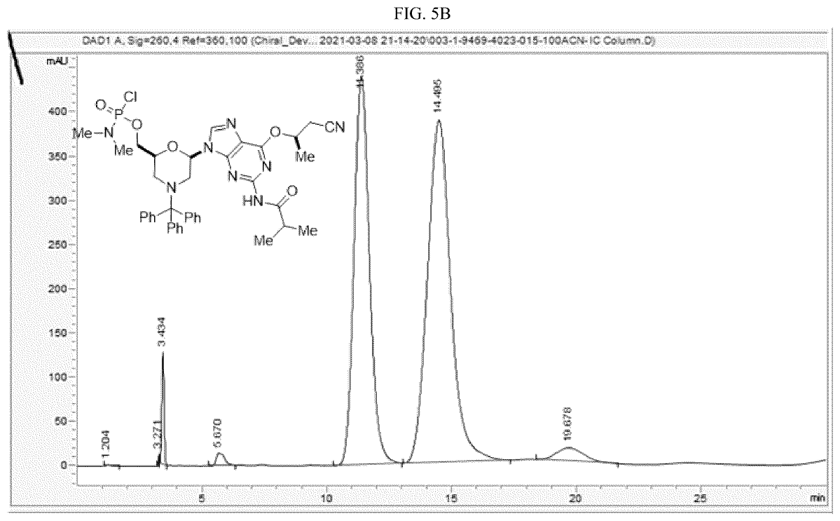

The structures of the bis-protected, activated guanine monomers described herein can also comprise stereoisomers of the Formulas I and II, in addition to the structures depicted in FIG. 1A and FIG. 1B. FIG. 5A and FIG. 5B demonstrate that the different isomers of the bis-protected, activated guanine monomers can be separated and isolated.

While the terms used herein are believed to be well understood by one of ordinary skill in the art, definitions are set forth herein to facilitate explanation of the subject matter disclosed herein.

Unless defined otherwise, all technical and scientific terms used herein have the same meaning as commonly understood by one of ordinary skill in the art to which the subject matter disclosed herein belongs. Although any methods, devices, and materials similar or equivalent to those described herein can be used in the practice or testing of the presently disclosed subject matter, representative methods, devices, and materials are described herein.

All combinations of method or process steps as used herein can be performed in any order, unless otherwise specified or clearly implied to the contrary by the context in which the referenced combination is made.

The methods and devices of the present disclosure, including components thereof, can comprise, consist of, or consist essentially of the essential elements and limitations of the embodiments described herein, as well as any additional or optional components or limitations described herein or otherwise useful.

Unless otherwise indicated, all numbers expressing physical dimensions, quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently disclosed subject matter.

“R” and “S” as terms describing isomers are descriptors of the stereochemical configuration at asymmetrically substituted atoms, including but not limited to: carbon, sulfur, phosphorous and quaternary nitrogen. The designation of asymmetrically substituted atoms as “R” or “S” is done by application of the Cahn-Ingold-Prelog priority rules, as are well known to those skilled in the art, and described in the International Union of Pure and Applied Chemistry (IUPAC) Rules for the Nomenclature of Organic Chemistry. Section E, Stereochemistry.

“Pharmaceutically acceptable salt” as used herein refers to acid addition salts or base addition salts of the compounds in the present disclosure. A pharmaceutically acceptable salt is any salt which retains the activity of the parent compound and does not impart any unduly deleterious or undesirable effect on a subject to whom it is administered and in the context in which it is administered. Pharmaceutically acceptable salts include, but are not limited to, metal complexes and salts of both inorganic and carboxylic acids. Pharmaceutically acceptable salts also include metal salts such as aluminum, calcium, iron, magnesium, manganese, sodium and complex salts. In addition, pharmaceutically acceptable salts include, but are not limited to, acid salts such as acetic, aspartic, alkylsulfonic, arylsulfonic, axetil, benzenesulfonic, benzoic, bicarbonic, bisulfuric, bitartaric, butyric, calcium edetate, camsylic, carbonic, chlorobenzoic, citric, edetic, edisylic, estolic, esyl, esylic, formic, fumaric, gluceptic, gluconic, glutamic, glycolic, glycolylarsanilic, hexamic, hexylresorcinoic, hydrabamic, hydrobromic, hydrochloric, hydroiodic, hydroxynaphthoic, isethionic, lactic, lactobionic, maleic, malic, malonic, mandelic, methanesulfonic, methylnitric, methylsulfuric, mucic, muconic, napsylic, nitric, oxalic, p nitromethanesulfonic, pamoic, pantothenic, phosphoric, monohydrogen phosphoric, dihydrogen phosphoric, phthalic, polygalactouronic, propionic, salicylic, stearic, succinic, sulfamic, sulfanlic, sulfonic, sulfuric, tannic, tartaric, teoclic, toluenesulfonic, and the like.

The term “pharmaceutical composition” includes preparations suitable for administration to mammals, e.g., humans. When the compounds of the present invention are administered as pharmaceuticals to mammals, e.g., humans, they can be given per se or as a pharmaceutical composition containing, for example, 0.1% to 99.9% (more preferably, 0.5 to 90%) of active ingredient in combination with a pharmaceutically acceptable carrier.

The term “alkyl” includes branched, straight chain and cyclic, substituted or unsubstituted saturated aliphatic hydrocarbon groups. Examples of C₁-C₆ alkyl groups include, but are not limited to, methyl, ethyl, propyl, isopropyl, butyl, sec-butyl, tert-butyl, pentyl, isopentyl, neopentyl, hexyl, isohexyl, cyclohexyl, cyclohexylmethyl, cyclopropylmethyl and neohexyl radicals.

The term “aryl” includes a 6- to 14-membered monocyclic, bicyclic or tricyclic aromatic hydrocarbon ring system. Examples of an aryl group include phenyl and naphthyl.

The halogen can be F, Cl, Br or I.

The term “cycloalkyl” includes a cycloalkyl ring containing 5 to 12 carbon atoms.

Examples include cyclopentyl, cyclohexyl, cycloheptyl, cyclooctyl, cyclodecyl and cyclododecyl.

### Examples

### Abbreviations

The following abbreviations may be used throughout the examples.
DBU: 1,8-Diazabicyclo[5.4.0]undec-7-ene
DCM: dichloromethane
DMAP: 4-(Dimethylamino)pyridine
DMF: N,N-Dimethylformamide
DMSO: Dimethyl sulfoxide
TEA: triethylamine
TFA: trifluoroacetic acid
THF Tetrahydrofuran
TBDPS: t-butyldiphenylsilyl
TBS: tert-butyldimethylsilyl
TBS-Cl: tert-butyldimethylsilyl chloride
Ph: phenyl
EA: ethyl acetate
ACN: acetonitrile

### Example 1: Synthesis of Bis-Protected, Activated Guanine Morpholino Monomers

### General Reaction Scheme

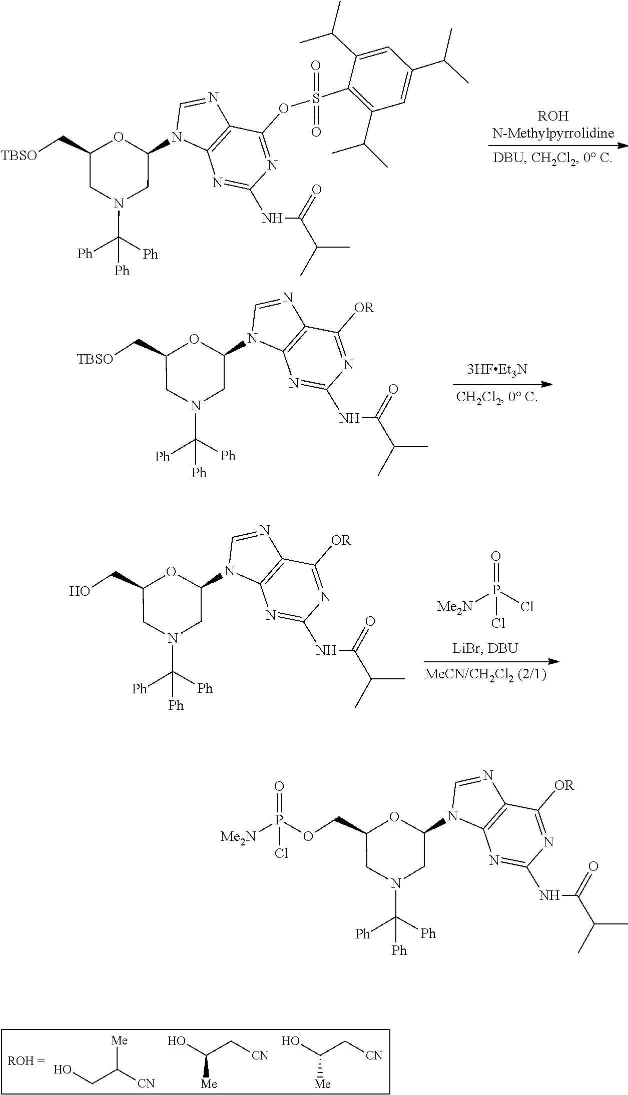

### Synthesis of N-(6-(2-cyanopropoxy)-9-((2R,6S)-6-(hydroxymethyl)-4-tritylmorpholin-2-yl)-9H-purin-2-yl)isobutyramide

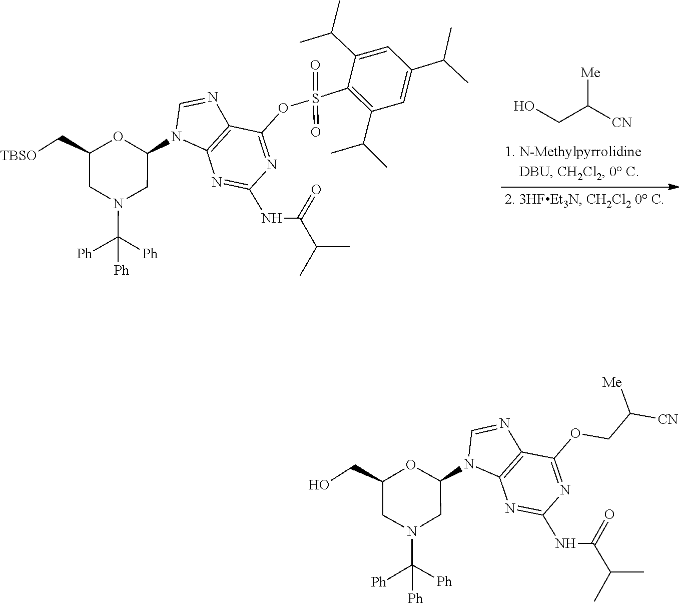 [SMILES: CC(C)C(=O)Nc1nc(OS(=O)(=O)c2c(C(C)C)cc(C(C)C)cc2C(C)C)c2ncn([C@H]3CN(C(c4ccccc4)(c4ccccc4)c4ccccc4)C[C@@H](CO[Si](C)(C)C(C)(C)C)O3)c2n1 ; CC(C#N)CO ; CC(C#N)COc1nc(NC(=O)C(C)C)nc2c1ncn2[C@H]1CN(C(c2ccccc2)(c2ccccc2)c2ccccc2)C[C@@H](CO)O1]

A solution of 9-((2R,6S)-6-(((tert-butyldimethylsilyl)oxy)methyl)-4-tritylmorpholin-2-yl)-2-isobutyramido-9H-purin-6-yl 2,4,6-triisopropylbenzenesulfonate (5.50 g, 5.73 mmol) (prepared by the procedure described in CA2813183) in CH₂Cl₂ (57.3 ml, 5.733 mmol) was and cooled to 0° C. and treated with 1-methylpyrrolidine (2.38 ml, 22.93 mmol) in CH₂Cl₂ (5.0 ml). After being stirred at 0° C. for 1 h, a solution of 3-hydroxy-2-methylpropanenitrile (1.95 g, 22.93 mmol) and DBU (1.12 ml, 7.45 mmol) in CH₂Cl₂ (5.0 ml) was added and continue stirring at 0° C. for 2-3 h (reaction was monitored by LCMS). The reaction mixture was diluted with 1.0 M aq. NaH₂PO₄ (50 ml) and water (50 ml) and stirred at rt for 30 min. The CH₂Cl₂ layer was separated and the aqueous layer was washed twice with CH₂Cl₂ (30 ml). The combined organic layer was washed with brine (30 ml), dried over Na₂SO₄, filtered, and concentrated in vacuo. The crude residue was purified by column chromatography (ethyl acetate in n-heptane=0% to 80%) to give the N-(9-((2R,6S)-6-(((tert-butyldimethylsilyl)oxy)methyl)-4-tritylmorpholin-2-yl)-6-(2-cyanopropoxy)-9H-purin-2-yl)isobutyramide (4.42 g, 5.816 mmol) contaminated with 3-hydroxy-2-methylpropanenitrile, which was used for next step without further purification.

The above obtained compound was dissolved in CH₂Cl₂ (58.2 ml, 5.81 mmol) in a plastic container and cooled to 0° C. Triethylamine trihydrofluoride (9.57 g, 58.15 mmol) was added to the reaction solution in drop-wise. The reaction solution was stirred at 0° C. for 7-8 h. The reaction mixture was poured into a ice-cold solution of sodium bicarbonate (7.33 g, 87.234 mmol) in water (60 ml) and stirred ar RT for 1 h. Then the CH₂Cl₂ layer was separated and the aqueous layer was extracted twice with CH₂Cl₂ (50 ml). The combined organic layer was dried over Na₂SO₄, filtered, concentrated under reduced pressure and purified by a silica gel column chromatography to give 2.59 g (4.01 mmol, 70%) of the title compound as white solid. ¹H NMR (400 MHz, CDCl₃) δ 7.88 (s, 1H), 7.82 (s, 1H), 7.58-7.44 (m, 6H), 7.33 (t, J=7.6 Hz, 6H), 7.26-7.16 (m, 3H), 6.28 (dd, J=9.9, 2.4 Hz, 1H), 4.80-4.68 (m, 1H), 4.66-4.52 (m, 1H), 4.41-4.30 (m, 1H), 3.64 (qd, J=11.8, 5.0 Hz, 2H), 3.47 (dt, J=11.4, 2.4 Hz, 1H), 3.41-3.28 (m, 1H), 3.22-3.14 (m, 1H), 3.14-3.10 (s, 1H), 1.84 (t, J=10.6 Hz, 1H), 1.62 (t, J=11.2 Hz, 1H), 1.49 (d, J=7.1 Hz, 3H), 1.39 (d, J=6.8 Hz, 3H), 1.36 (d, J=6.8 Hz, 3H).

### Synthesis of N-(6-(((R)-1-cyanopropan-2-yl)oxy)-9-((2R,6S)-6-(hydroxymethyl)-4-tritylmorpholin-2-yl)-9H-purin-2-yl)isobutyramide

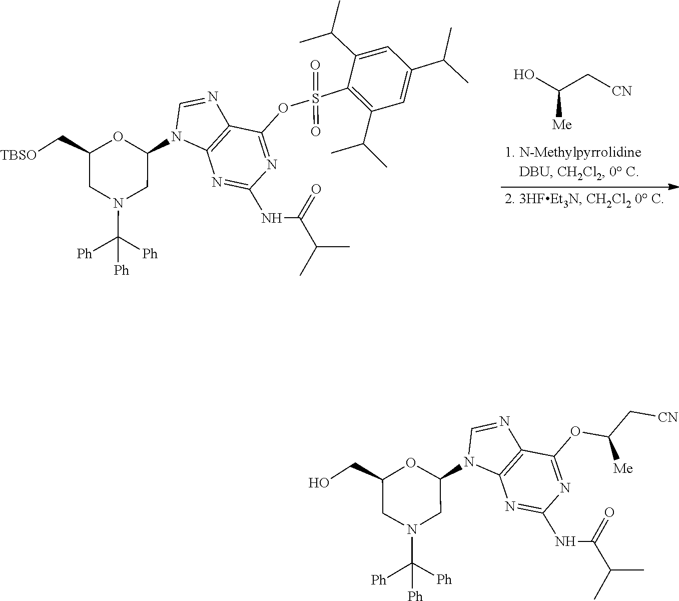 [SMILES: CC(C)C(=O)Nc1nc(OS(=O)(=O)c2c(C(C)C)cc(C(C)C)cc2C(C)C)c2ncn([C@H]3CN(C(c4ccccc4)(c4ccccc4)c4ccccc4)C[C@@H](CO[Si](C)(C)C(C)(C)C)O3)c2n1 ; C[C@@H](O)CC#N ; CC(C)C(=O)Nc1nc(O[C@H](C)CC#N)c2ncn([C@H]3CN(C(c4ccccc4)(c4ccccc4)c4ccccc4)C[C@@H](CO)O3)c2n1]

Using the same procedure and same volumes as for the preparation of N-(6-(2-cyanopropoxy)-9-((2R,6S)-6-(hydroxymethyl)-4-tritylmorpholin-2-yl)-9H-purin-2-yl)isobutyramide, 20.0 g of 9-((2R,6S)-6-(((tert-butyldimethylsilyl)oxy)methyl)-4-tritylmorpholin-2-yl)-2-isobutyramido-9H-purin-6-yl 2,4,6-triisopropylbenzenesulfonate gave 8.5 g of the title compound (13.16 mmol, 63%) as a white solid.

¹H NMR (400 MHz, CDCl₃) δ 7.83 (s, 1H), 7.82 (s, 1H), 7.57-7.43 (m, 6H), 7.33 (t, J=7.6 Hz, 6H), 7.26-7.18 (m, 3H), 6.27 (dd, J=9.9, 2.3 Hz, 1H), 5.73-5.62 (m, 1H), 4.41-4.31 (m, 1H), 3.70-3.56 (m, 2H), 3.51-3.43 (m, 1H), 3.22-3.14 (m, 1H), 3.08 (s, 1H), 2.97 (dd, J=5.9, 2.5 Hz, 2H), 1.86 (t, J=10.6 Hz, 2H), 1.66-1.57 (m, 4H), 1.39 (d, J=6.9 Hz, 3H), 1.36 (d, J=6.8 Hz, 3H).

### Synthesis of N-(6-(((S)-1-cyanopropan-2-yl)oxy)-9-((2R,6S)-6-(hydroxymethyl)-4-tritylmorpholin-2-yl)-9H-purin-2-yl)isobutyramide

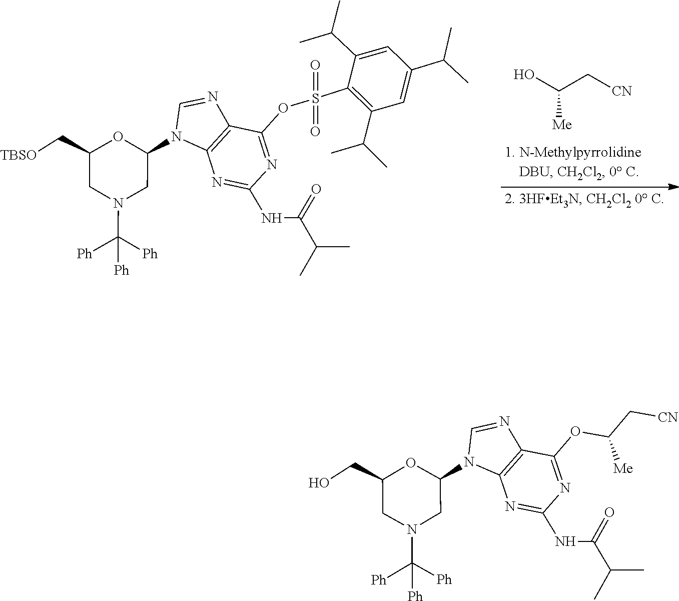 [SMILES: CC(C)C(=O)Nc1nc(OS(=O)(=O)c2c(C(C)C)cc(C(C)C)cc2C(C)C)c2ncn([C@H]3CN(C(c4ccccc4)(c4ccccc4)c4ccccc4)C[C@@H](CO[Si](C)(C)C(C)(C)C)O3)c2n1 ; C[C@H](O)CC#N ; CC(C)C(=O)Nc1nc(O[C@@H](C)CC#N)c2ncn([C@H]3CN(C(c4ccccc4)(c4ccccc4)c4ccccc4)C[C@@H](CO)O3)c2n1]

Using the same procedure and same volumes as for the preparation of N-(6-(2-cyanopropoxy)-9-((2R,6S)-6-(hydroxymethyl)-4-tritylmorpholin-2-yl)-9H-purin-2-yl)isobutyramide, 3.00 g of 9-((2R,6S)-6-(((tert-butyldimethylsilyl)oxy)methyl)-4-tritylmorpholin-2-yl)-2-isobutyramido-9H-purin-6-yl 2,4,6-triisopropylbenzenesulfonate gave 1.38 g of the title compound (1.87 mmol, 68%) as a white solid.

¹H NMR (400 MHz, CDCl₃) δ 7.88 (s, 1H), 7.82 (s, 1H), 7.58-7.42 (m, 6H), 7.33 (t, J=7.6 Hz, 6H), 7.24-7.18 (m, 3H), 6.27 (dd, J=9.9, 2.4 Hz, 1H), 5.74-5.62 (m, 1H), 4.40-4.30 (m, 1H), 3.71-3.56 (m, 2H), 3.52-3.43 (m, 1H), 3.22-3.13 (m, 1H), 3.08 (s, 1H), 3.00 (dd, J=16.9, 6.1 Hz, 1H), 2.92 (dd, J=16.8, 5.5 Hz, 1H), 1.83 (t, J=10.6 Hz, 1H), 1.67-1.56 (m, 4H), 1.38 (t, J=7.2 Hz, 6H).

### Synthesis of ((2S,6R)-6-(6-(2-cyanopropoxy)-2-isobutyramido-9H-purin-9-yl)-4-tritylmorpholin-2-yl)methyl dimethylphosphoramidochloridate

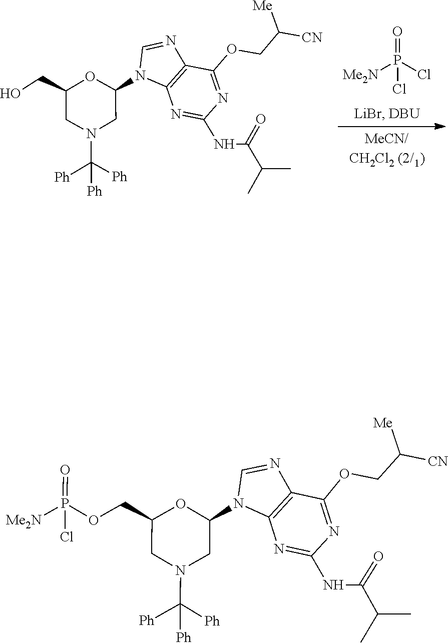 [SMILES: CC(C#N)COc1nc(NC(=O)C(C)C)nc2c1ncn2[C@H]1CN(C(c2ccccc2)(c2ccccc2)c2ccccc2)C[C@@H](CO)O1 ; CN(C)P(=O)(Cl)Cl ; CC(C#N)COc1nc(NC(=O)C(C)C)nc2c1ncn2[C@H]1CN(C(c2ccccc2)(c2ccccc2)c2ccccc2)C[C@@H](COP(=O)(Cl)N(C)C)O1]

To a solution of N-(6-(2-cyanopropoxy)-9-((2R,6S)-6-(hydroxymethyl)-4-tritylmorpholin-2-yl)-9H-purin-2-yl)isobutyramide (890 mg, 1.37 mmol) in CH₃CN (7990 μl, 152.98 mmol)/CH₂Cl₂ (7981 μl, 124.03 mmol) was added lithium bromide (395 mg, 4.54 mmol) at room temperature and stirred until a clear solution was obtained. Then, the reaction mixture was cooled to 0° C. and was added a solution of DBU (686 μl, 4.54 mmol) in CH₃CN (1.0 ml) followed by addition of N,N-Dimethylphosphoramic dichloride (262 μl, 2.205 mmol) in CH₃CN (1.0 ml) at 0° C. After 2 h, the reaction was quenched with 10% aq. Citric acid solution (20 ml) and diluted with ethylacetate (30 ml). After 30 min, ethylacetate layer was separated and the aqueous layer was extracted twice with ethylacetate (30 ml). The combined organic layer was eashed with water, brine, dried over Na₂SO₄, filtered and concentrated under reduced vaccum, The crude residue was purified by silica gel column to give 820 mg (1.06 mmol, 77%) of title compound.

### Preparative HPLC Conditions:

Column: Chiralpak IA, 21×250 mm 5 u
Flowrate: 20 m/min
Mobile Phase: 40% Heptane 60% EA
Gradient: Isocratic
Runtime 20 mins
Injection Volume: 500 uL
Detection: 260 nm

### ((2S,6R)-6-(6-(2-cyanopropoxy)-2-isobutyramido-9H-purin-9-yl)-4-tritylmorpholin-2-yl)methyl(S)-dimethylphosphoramidochloridate

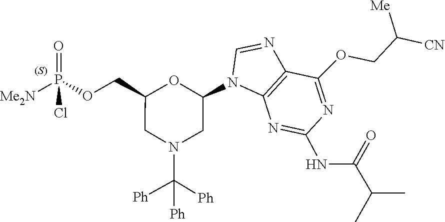

Under HPLC conditions, the titled compound gave two peaks due to methyl stereochemistry adjacent to cyano group.

### Peak 1 (10.20 Min):

¹H NMR (400 MHz, Acetone-d6) δ 9.17 (s, 1H), 7.94 (s, 1H), 7.53-7.34 (m, 6H), 7.22 (t, J=7.6 Hz, 6H), 7.08 (t, J=7.4 Hz, 3H), 6.27 (dd, J=9.9, 2.4 Hz, 1H), 4.61-4.45 (m, 3H), 4.06 (dd, J=8.5, 5.0 Hz, 2H), 3.48-3.37 (m, 2H), 3.21 (d, J=11.7 Hz, 1H), 3.09-2.97 (m, 1H), 2.48 (s, 3H), 2.45 (s, 3H), 1.87-1.83 (m, 1H), 1.55 (t, J=11.2 Hz, 1H), 1.30 (d, J=7.1 Hz, 3H), 1.13 (d, J=5.1 Hz, 3H), 1.10 (d, J=7.1 Hz, 3H); ³¹P NMR (162 MHz, Acetone-d6) δ 17.53.

### Peak 2 (11.35 Min):

¹H NMR (400 MHz, Acetone-d6) δ 9.19 (s, 1H), 7.94 (s, 1H), 7.55-7.35 (m, 6H), 7.21 (t, J=7.7 Hz, 6H), 7.07 (t, J=7.4 Hz, 3H), 6.27 (dd, J=9.9, 2.4 Hz, 1H), 4.61-4.48 (m, 3H), 4.05 (dd, J=8.5, 5.0 Hz, 2H), 3.47-3.34 (m, 2H), 3.21 (dt, J=12.0, 2.5 Hz, 1H), 3.10-2.99 (m, 1H), 2.48 (s, 3H), 2.44 (s, 3H), 1.91-1.80 (m, 1H), 1.55 (t, J=11.2 Hz, 1H), 1.29 (d, J=7.1 Hz, 3H), 1.15 (d, J=6.8 Hz, 3H), 1.12 (d, J=6.8 Hz, 3H); ³¹P NMR (162 MHz, Acetone-d6) δ 17.56.

### ((2S,6R)-6-(6-(2-cyanopropoxy)-2-isobutyramido-9H-purin-9-yl)-4-tritylmorpholin-2-yl)methyl(R)-dimethylphosphoramidochloridate

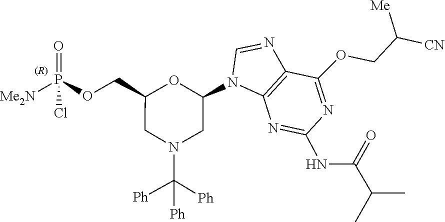

Under HPLC conditions, the titled compound gave two peaks due to methyl stereochemistry adjacent to cyano group.

### Peak 3 (12.77 Min):

¹H NMR (400 MHz, Acetone-d6) δ 9.18 (s, 1H), 7.95 (s, 1H), 7.54-7.33 (m, 6H), 7.21 (t, J=7.7 Hz, 6H), 7.08 (t, J=7.4 Hz, 3H), 6.26 (dd, J=9.9, 2.4 Hz, 1H), 4.60-4.44 (m, 3H), 4.15-4.03 (m, 2H), 3.49-3.36 (m, 2H), 3.20 (dt, J=11.8, 2.4 Hz, 1H), 3.10-2.99 (m, 1H), 2.48 (s, 3H), 2.45 (s, 3H), 1.91-1.79 (m, 1H), 1.59 (t, J=11.2 Hz, 1H), 1.29 (d, J=7.1 Hz, 3H), 1.15 (d, J=6.8 Hz, 3H), 1.12 (d, J=6.8 Hz, 3H); ³¹P NMR (162 MHz, Acetone-d6) δ 17.17.

### Peak 4 (14.36 Min):

¹H NMR (400 MHz, Acetone-d6) δ 9.17 (s, 1H), 7.95 (s, 1H), 7.52-7.31 (m, 6H), 7.21 (t, J=7.7 Hz, 6H), 7.08 (t, J=7.4 Hz, 3H), 6.26 (dd, J=9.9, 2.4 Hz, 1H), 4.91 (s, 1H), 4.60-4.47 (m, 3H), 4.15-4.03 (m, 2H), 3.47-3.34 (m, 2H), 3.20 (dt, J=11.8, 2.4 Hz, 1H), 3.10-2.99 (m, 1H), 2.48 (s, 3H), 2.45 (s, 3H), 1.90-1.78 (m, 1H), 1.59 (t, J=11.2 Hz, 1H), 1.29 (d, J=7.1 Hz, 3H), 1.15 (d, J=6.9 Hz, 3H), 1.12 (d, J=6.8 Hz, 3H); ³¹P NMR (162 MHz, Acetone-d6) δ 17.17.

### Synthesis of ((2S,6R)-6-(6-(((R)-1-cyanopropan-2-yl)oxy)-2-isobutyramido-9H-purin-9-yl)-4-tritylmorpholin-2-yl)methyl dimethylphosphoramidochloridate

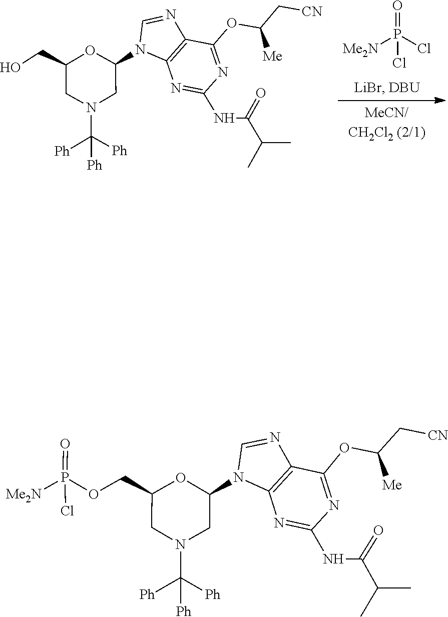 [SMILES: CC(C)C(=O)Nc1nc(O[C@H](C)CC#N)c2ncn([C@H]3CN(C(c4ccccc4)(c4ccccc4)c4ccccc4)C[C@@H](CO)O3)c2n1 ; CN(C)P(=O)(Cl)Cl ; CC(C)C(=O)Nc1nc(O[C@H](C)CC#N)c2ncn([C@H]3CN(C(c4ccccc4)(c4ccccc4)c4ccccc4)C[C@@H](COP(=O)(Cl)N(C)C)O3)c2n1]

Using the same procedure as for the preparation of ((2S,6R)-6-(6-(2-cyanopropoxy)-2-isobutyramido-9H-purin-9-yl)-4-tritylmorpholin-2-yl)methyl dimethyl-phosphoramidochloridate, 3.3 g of N-(6-(((R)-1-cyanopropan-2-yl)oxy)-9-((2R,6S)-6-(hydroxymethyl)-4-tritylmorpholin-2-yl)-9H-purin-2-yl)isobutyramide gave 2.56 g of the title compound (3.32 mmol, 65% yield).

### Preparative HPLC Conditions:

Column: Chiralpak IC, 30×250 mm 5 u
Flowrate: 30 mL/min
Mobile Phase: 100% ACN
Gradient: Isocratic
Runtime 32 mins
Injection Volume: 500 uL
Detection: 260 nm

### ((2S,6R)-6-(6-(((R)-1-cyanopropan-2-yl)oxy)-2-isobutyramido-9H-purin-9-yl)-4-tritylmorpholin-2-yl)methyl(S)-dimethylphosphoramidochloridate

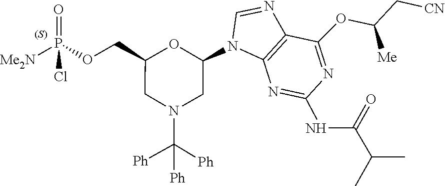

### Retention Time: 11.39 Min

¹H NMR (400 MHz, Acetone-d6) δ 9.22 (s, 1H), 7.93 (s, 1H), 7.54-7.33 (m, 6H), 7.21 (t, J=7.7 Hz, 6H), 7.12-7.03 (m, 3H), 6.25 (dd, J=9.9, 2.4 Hz, 1H), 5.56-5.44 (m, 1H), 4.59-4.49 (m, 1H), 4.09-4.01 (m, 2H), 3.40 (dt, J=11.6, 2.5 Hz, 1H), 3.21 (dt, J=12.0, 2.5 Hz, 1H), 3.07 (dd, J=17.1, 5.4 Hz, 1H), 3.04-2.98 (m, 1H), 2.93 (dd, J=17.1, 5.2 Hz, 1H), 2.47 (s, 3H), 2.44 (s, 3H), 1.90-1.82 (m, 1H), 1.54 (t, J=11.2 Hz, 1H), 1.42 (d, J=6.3 Hz, 3H), 1.14 (d, J=6.9 Hz, 4H), 1.11 (d, J=6.8 Hz, 3H); ³¹P NMR (162 MHz, Acetone-d6) δ 17.56.

### ((2S,6R)-6-(6-(((R)-1-cyanopropan-2-yl)oxy)-2-isobutyramido-9H-purin-9-yl)-4-tritylmorpholin-2-yl)methyl(R)-dimethylphosphoramidochloridate

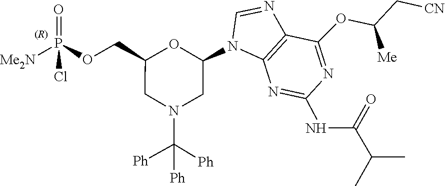

### Retention Time: 14.50 Min

¹H NMR (400 MHz, Acetone-d6) δ 9.22 (s, 1H), 7.94 (s, 1H), 7.51-7.36 (m, 6H), 7.21 (t, J=7.7 Hz, 6H), 7.11-7.03 (m, 3H), 6.25 (dd, J=9.8, 2.4 Hz, 1H), 5.56-5.44 (m, 1H), 4.58-4.48 (m, 1H), 4.16-4.00 (m, 2H), 3.40 (dt, J=11.5, 2.5 Hz, 1H), 3.20 (dt, J=11.9, 2.5 Hz, 1H), 3.07 (dd, J=17.1, 5.4 Hz, 1H), 3.04-2.98 (m, OH), 2.93 (dd, J=17.1, 5.2 Hz, 1H), 2.48 (s, 3H), 2.44 (s, 3H), 1.90-1.83 (m, 1H), 1.58 (t, J=11.2 Hz, 1H), 1.42 (d, J=6.3 Hz, 3H), 1.15 (d, J=6.9 Hz, 4H), 1.12 (d, J=6.8 Hz, 3H); ³¹P NMR (162 MHz, Acetone-d6) δ 17.18.

### Synthesis of ((2S,6R)-6-(6-(((S)-1-cyanopropan-2-yl)oxy)-2-isobutyramido-9H-purin-9-yl)-4-tritylmorpholin-2-yl)methyl dimethylphosphoramidochloridate

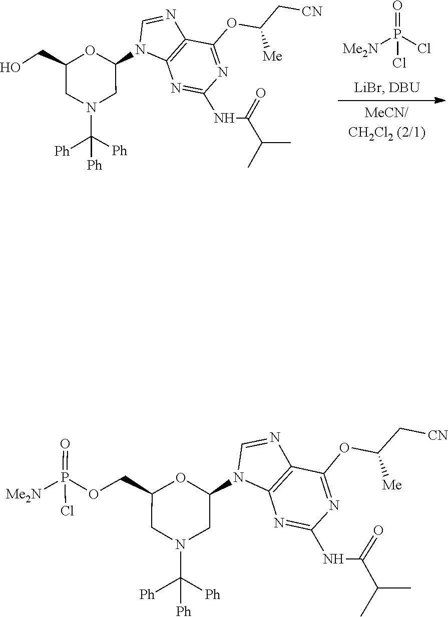

Using the same procedure as for the preparation of ((2S,6R)-6-(6-(2-cyanopropoxy)-2-isobutyramido-9H-purin-9-yl)-4-tritylmorpholin-2-yl)methyl dimethyl-phosphoramidochloridate, 1.0 g of N-(6-(((S)-1-cyanopropan-2-yl)oxy)-9-((2R,6S)-6-(hydroxymethyl)-4-tritylmorpholin-2-yl)-9H-purin-2-yl)isobutyramide gave 0.810 g of the title compound (3.32 mmol, 68% yield).

### Example 2: Synthesis of a Bis-Protected, Activated Guanine Deoxyribonucleoside

### Synthesis of N-(9-((2R,4S,5R)-4-((tert-butyldimethylsilyl)oxy)-5-(((tert-butyldimethylsilyl)oxy)methyl)tetrahydrofuran-2-yl)-6-oxo-6,9-dihydro-1H-purin-2-yl)isobutyramide

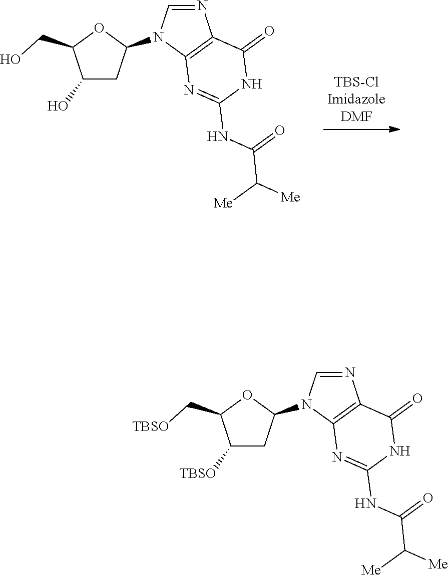 [SMILES: CC(C)C(=O)Nc1nc2c(ncn2[C@H]2C[C@H](O)[C@@H](CO)O2)c(=O)[nH]1 ; CC(C)C(=O)Nc1nc2c(ncn2[C@H]2C[C@H](O[Si](C)(C)C(C)(C)C)[C@@H](CO[Si](C)(C)C(C)(C)C)O2)c(=O)[nH]1]

N-(9-((2R,4S,5R)-4-hydroxy-5-(hydroxymethyl)tetrahydrofuran-2-yl)-6-oxo-6,9-dihydro-1H-purin-2-yl)isobutyramide (2.5 g, 7.411 mmol) was co-evaporated with anhydrous pyridine once before it was dissolved in DMF (25 mL) in a flask, into which was added imidazole (2.52 g, 37.055 mmol) followed by addition of TBS-Cl (2.79 g, 18.528 mmol) portionwise at room temperature. The reaction mixture was kept stirring at room temperature for 48 hr. Into the mixture was then added water (200 mL), and the solid precipitate was collected and rinsed with water. The solid was redissolved in DCM, was washed subsequently with sat. sodium bicarbonate (aq.) and half sat. brine, dried over Na₂SO₄ and concentrated. Silica gel column purification of the concentrate with MeOH in DCM afforded 4.11 g of product. ¹H NMR (400 MHz, CHLOROFORM-d) δ ppm 11.95 (s, 1H), 8.18 (s, 1H), 7.96 (s, 1H), 6.22 (dd, J=6.4, 6.8 Hz, 1H), 4.52-4.61 (m, 1H), 3.98 (m, 1H), 3.76 (d, J=3.2 Hz, 2H), 2.61 (m, 1H), 2.28-2.51 (m, 2H), 1.27 (br d, J=7.0 Hz, 3H), 1.28 (br d, J=6.8 Hz, 3H), 0.91 (s, 9H), 0.90 (s, 9H), 0.10 (s, 6H), 0.07 (s, 3H), 0.07 (s, 3H).

MS (ESI) m/z: calculated for C₂₆H₄₇N₅O₅Si₂ [M+H]⁺: 566.3; found 566.2.

### Synthesis of N-(9-((2R,4S,5R)-4-((tert-butyldimethylsilyl)oxy)-5-(((tert-butyldimethylsilyl)oxy)methyl)tetrahydrofuran-2-yl)-6-(((R)-1-cyanopropan-2-yl)oxy)-9H-purin-2-yl)isobutyramide

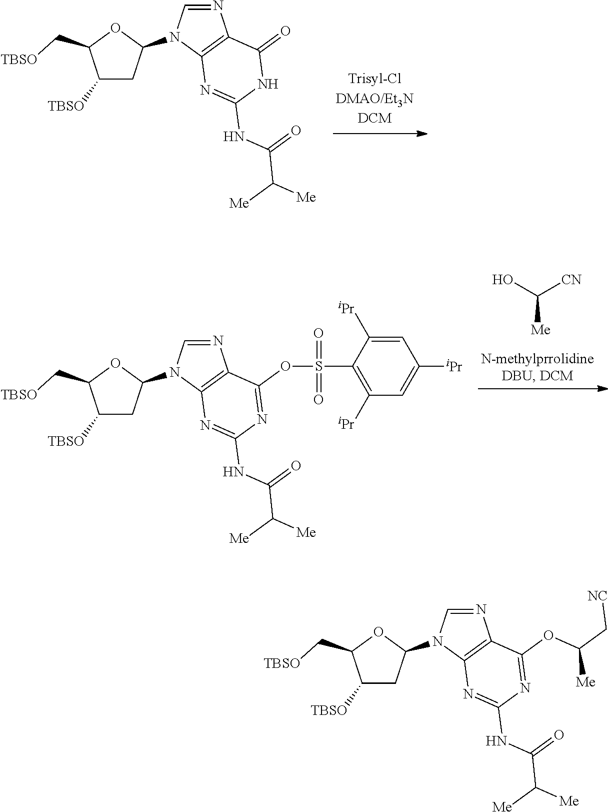 [SMILES: CC(C)C(=O)Nc1nc2c(ncn2[C@H]2C[C@H](O[Si](C)(C)C(C)(C)C)[C@@H](CO[Si](C)(C)C(C)(C)C)O2)c(=O)[nH]1 ; CC(C)C(=O)Nc1nc(OS(=O)(=O)c2c(C(C)C)cc(C(C)C)cc2C(C)C)c2ncn([C@H]3C[C@H](O[Si](C)(C)C(C)(C)C)[C@@H](CO[Si](C)(C)C(C)(C)C)O3)c2n1 ; C[C@@H](O)C#N ; CC(C)C(=O)Nc1nc(O[C@H](C)CC#N)c2ncn([C@H]3C[C@H](O[Si](C)(C)C(C)(C)C)[C@@H](CO[Si](C)(C)C(C)(C)C)O3)c2n1]

N-(9-((2R,4S,5R)-4-((tert-butyldimethylsilyl)oxy)-5-(((tert-butyldimethylsilyl)oxy)methyl)tetrahydrofuran-2-yl)-6-oxo-6,9-dihydro-1H-purin-2-yl)isobutyramide (4.11 g, 7.263 mmol) was co-evaporated with anhydrous MeCN twice before it was dissolved in DCM (41.1 mL) in a flask at room temperature, into which was added DMAP (0.089 g, 0.726 mmol) and triethylamine (3.04 mL, 21.79 mmol), followed by addition of 2,4,6-triisopropylbenzenesulfonyl chloride (3.30 g, 10.895 mmol). The reaction mixture was stirred at room temperature for 20 hr before it was cooled in an ice bath, and quenched with sodium dihydrogen phosphate aqueous solution (105 mL, 87.159 mmol, 10 wt %). After phase separation, it was back extracted multiple times with DCM (100 mL). The combined DCM layers were washed with brine (10 wt %), dried over Na₂SO₄, and concentrated.

The residue was co-evaporated with anhydrous toluene three times before it was redissolved in DCM (60.4 mL, 938.783 mmol) in a flask in an ice bath, into which was added N-methylpyrrolidine (1.509 mL, 14.515 mmol). The mixture was first stirred at 0° C. for 1 hr and then stirred at ambient temperature for another before it was cooled back to 0° C. Into the mixture was then added a solution of (R)-3-hydroxybutanenitrile (0.772 g, 9.072 mmol) and DBU (1.367 mL, 9.072 mmol) in DCM (6 mL) via a cannula. It was kept stirring at 0° C. for 4 hr and at ambient temperature for 1 hr before additional (R)-3-hydroxybutanenitrile (0.579 g, 6.804 mmol) and DBU (0.273 mL, 1.814 mmol) was added. It was stirred at ambient temperature for 1 hr before it was quenched with sodium dihydrogen phosphate (174 mL, 145.147 mmol) (aq. 10 wt %) at 0° C. The mixture was extracted with DCM multiple times (150 mL×2). The combined DCM layers were washed subsequently with water (80 mL×2) and brine (80 mL, 10%), dried over Na₂SO₄, and concentrated. Silica gel column purification of the concentrate with heptane-ethyl acetate afforded 1.58 g of product.

¹H NMR (400 MHz, CHLOROFORM-d) δ ppm 8.16 (s, 1H), 7.79 (s, 1H), 6.39 (dd, J=6.8, 6.4 Hz, 1H), 5.67 (m, 1H), 4.60 (m, 1H), 4.00 (m, 1H), 3.85 (dd, J=11.2, 4.0 Hz, 1H), 3.77 (dd, J=11.2, 2.8 Hz, 1H), 3.05 (br s, 1H), 2.96 (d, J=5.8 Hz, 2H), 2.57 (m, 1H), 2.40 (ddd, J=13.0, 6.1, 3.9 Hz, 1H), 1.64 (d, J=6.5 Hz, 3H), 1.29 (d, J=7.0 Hz, 6H), 0.92 (s, 9H), 0.91 (s, 9H), 0.10 (s, 6H), 0.09 (s, 6H).

MS (ESI) m/z: calculated for C₃₀H₅₂N₆O₅Si₂ [M+H]⁺: 633.4; found 633.4.

### Synthesis of N-(9-((2R,4S,5R)-4-((tert-butyldimethylsilyl)oxy)-5-(hydroxymethyl)tetrahydrofuran-2-yl)-6-(((R)-1-cyanopropan-2-yl)oxy)-9H-purin-2-yl)isobutyramide

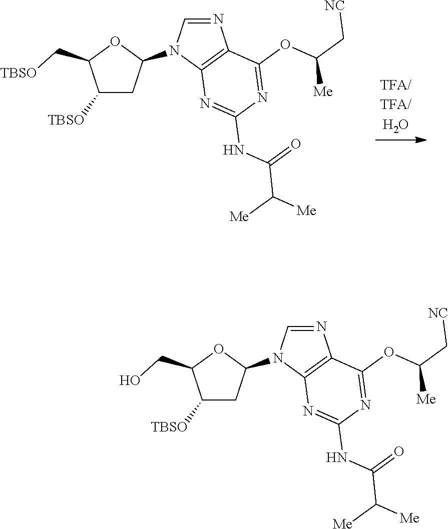 [SMILES: CC(C)C(=O)Nc1nc(O[C@H](C)CC#N)c2ncn([C@H]3C[C@H](O[Si](C)(C)C(C)(C)C)[C@@H](CO[Si](C)(C)C(C)(C)C)O3)c2n1 ; CC(C)C(=O)Nc1nc(O[C@H](C)CC#N)c2ncn([C@H]3C[C@H](O[Si](C)(C)C(C)(C)C)[C@@H](CO)O3)c2n1]

To a mixture of N-(9-((2R,4S,5R)-4-((tert-butyldimethylsilyl)oxy)-5-(((tert-butyldimethylsilyl)oxy)methyl)tetrahydrofuran-2-yl)-6-(((R)-1-cyanopropan-2-yl)oxy)-9H-purin-2-yl)isobutyramide (1.58 g, 2.496 mmol) in THF (10.64 mL, 129.907 mmol) and water (3.55 mL, 196.958 mmol) was added TFA (0.577 mL, 7.489 mmol) at 0° C. It was stirred in an ice bath for 5 hr. Into the mixture was added water (50 mL), and the solid precipitate was collected by filtration, and rinsed with water (30 mL×3). The solid was suspended in MeCN (50 mL), and sonicated for 5 min. The solid was collected by filtration, and the filtrate was concentrated and subjected to silica gel column chromatography, eluted with DCM-MeOH to give additional product fractions. The combined product fractions was co-evaporated with pyridine once and then anhydrous MeCN twice to give total 1.13 g of product.

¹H NMR (400 MHz, CD₃OD) δ ppm 8.41 (s, 1H), 6.48 (dd, J=6.8, 6.4 Hz, 1H), 5.72 (m, 1H), 4.79 (m, 1H), 3.97 (m, 1H), 3.79 (dd, J=12.0, 4.0 Hz, 1H), 3.74 (dd, J=12.0, 4.4 Hz, 1H), 3.17 (dd, J=17.2, 5.2 Hz, 1H), 3.04 (dd, J=17.2, 5.6 Hz, 1H), 2.81 (m, 2H), 2.44 (m, 1H), 1.60 (d, J=6.4 Hz, 3H), 1.23 (d, J=6.8 Hz, 6H), 0.95 (s, 9H), 0.17 (s, 3H), 0.15 (s, 3H).

MS (ESI) m/z: calculated for C₂₄H₃₈N₆O₅Si [M+H]⁺: 519.3; found 519.4.

### Synthesis of ((2R,3S,5R)-3-((tert-butyldimethylsilyl)oxy)-5-(6-(((R)-1-cyanopropan-2-yl)oxy)-2-isobutyramido-9H-purin-9-yl)tetrahydrofuran-2-yl)methyl dimethylphosphoramidochloridate

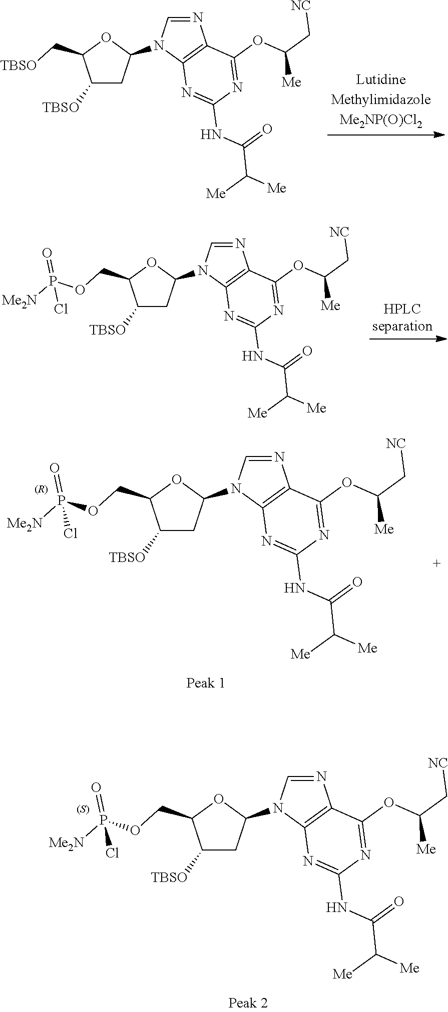

Into a suspension of N-(9-((2R,4S,5R)-4-((tert-butyldimethylsilyl)oxy)-5-(hydroxymethyl)tetrahydrofuran-2-yl)-6-(((R)-1-cyanopropan-2-yl)oxy)-9H-purin-2-yl)isobutyramide (1.134 g, 2.186 mmol) in DCM (18.99 mL, 295.147 mmol) and acetonitrile (18.96 mL, 362.922 mmol) was added 1-methylimidazole (0.105 mL, 1.312 mmol) and 2,6-lutidine (1.528 mL, 13.118 mmol), followed by addition of dimethylphosphoramidic dichloride (0.781 mL, 6.559 mmol) at rt. The suspension was then stirred at room temperature for 1 day before it was quenched with aqueous citric acid solution (88 mL, 45.912 mmol, 10%) at 0° C. It was extracted with DCM (113 mL×2). The combined DCM layers were washed subsequently with water twice and half sat. brine, dried over Na₂SO₄, and concentrated. Silica gel column purification of the concentrate with heptane-ethyl acetate afforded 944 mg of a stereoisomer mixture. MS (ESI) m/z: calculated for C₂₆H₄₃ClN₇O₆PSi [M+H]⁺: 644.3; found 644.2.

The stereoisomer mixture was subjected to the following HPLC separation method to isolate the (R) and (S)-stereoisomers of the product:

### HPLC Separation Method

Column: Chiralpak IC, 30×250 mm 5 u
Flowrate: 30 m/min
Mobile Phase: 50% MTBE 50% EA
Gradient: Isocratic
Runtime 17 mins
Injection Volume: 500 uL 40 mg/mL concentration
Detection: 260 nm

### Peak 1, Retention Time at 5.54 Min

### ((2R,3S,5R)-3-((tert-butyldimethylsilyl)oxy)-5-(6-(((R)-1-cyanopropan-2-yl)oxy)-2-isobutyramido-9H-purin-9-yl)tetrahydrofuran-2-yl)methyl(R)-dimethylphosphoramidochloridate

¹H NMR (400 MHz, CHLOROFORM-d) δ ppm 8.73 (br s, 1H), 7.90 (s, 1H), 6.31 (dd, J=6.8, 6.8 Hz, 1H), 5.67 (m, 1H), 4.76-4.91 (m, 2H), 4.11-4.27 (m, 2H), 3.05-3.29 (m, 2H), 2.94 (d, J=17.2, 5.2 Hz, 1H), 2.84 (m, 1H), 2.76 (s, 3H), 2.72 (s, 3H), 2.37 (ddd, J=13.6, 6.8, 2.9 Hz, 1H), 1.65 (d, J=6.3 Hz, 3H), 1.27 (d, J=6.8 Hz, 6H), 0.92 (s, 9H), 0.14 (s, 3H), 0.13 (s, 3H). ³¹P NMR (162 MHz, CHLOROFORM-d) δ ppm 19.17 (s, 1P).

### Peak 2, Rt at 9.09 Min

### ((2R,3S,5R)-3-((tert-butyldimethylsilyl)oxy)-5-(6-(((R)-1-cyanopropan-2-yl)oxy)-2-isobutyramido-9H-purin-9-yl)tetrahydrofuran-2-yl)methyl(S)-dimethylphosphoramidochloridate

¹H NMR (400 MHz, CHLOROFORM-d) δ ppm 8.57 (s, 1H), 7.90 (s, 1H), 6.32 (dd, J=7.2, 6.8 Hz, 1H), 5.65 (m, 1H), 4.74-4.83 (m, 1H), 4.54-4.74 (m, 1H), 4.35-4.49 (m, 1H), 4.18-4.28 (m, 1H), 3.17 (dd, J=16.8, 6.0 Hz, 1H), 3.06 (m, 1H), 2.95 (dd, J=17.0, 4.8 Hz, 1H), 2.79-2.88 (m, 1H), 2.69 (s, 3H), 2.65 (s, 3H), 2.35 (ddd, J=13.4, 6.4, 2.6 Hz, 1H), 1.65 (d, J=6.5 Hz, 3H), 1.22-1.34 (m, 6H), 0.93 (s, 9H), 0.15 (s, 3H), 0.13 (s, 3H). ³¹P NMR (162 MHz, CHLOROFORM-d) δ ppm 19.27 (s, 1P).

### Example 3: Synthesis of Pivalate Bis-Protected, Activated Guanine Deoxyribonucleoside

### Synthesis of 4-(((9-((2R,4S,5R)-4-((tert-butyldimethylsilyl)oxy)-5-(((tert-butyldimethylsilyl)oxy)methyl)tetrahydrofuran-2-yl)-2-isobutyramido-9H-purin-6-yl)oxy)methyl)phenyl pivalate

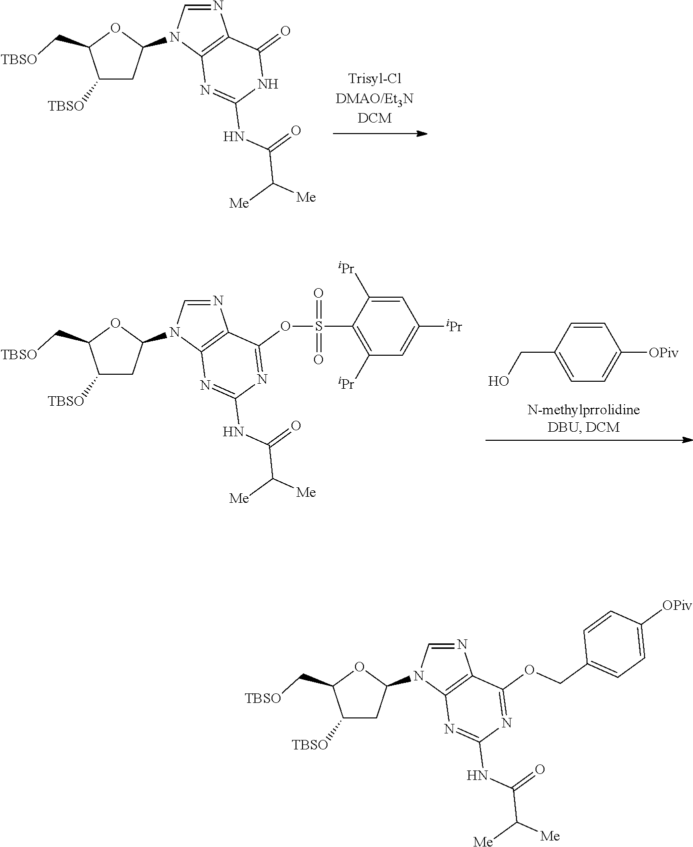

N-(9-((2R,4S,5R)-4-((tert-butyldimethylsilyl)oxy)-5-(((tert-butyldimethylsilyl)oxy)methyl)tetrahydrofuran-2-yl)-6-oxo-6,9-dihydro-1H-purin-2-yl)isobutyramide (1.50 g, 2.651 mmol) was co-evaporated with anhydrous pyridine and then with anhydrous MeCN before it was dissolved in DCM (15.00 mL, 233.126 mmol) in a flask with a room temperature water bath. Into the solution was added DMAP (0.032 g, 0.265 mmol) and triethylamine (1.108 mL, 7.952 mmol), followed by addition of 2,4,6-triisopropylbenzenesulfonyl chloride (1.445 g, 4.771 mmol). It was stirred for 1.5 hr before it was cooled in ice bath and quenched with sodium dihydrogen phosphate aqueous solution (54.9 mL, 39.762 mmol, 10 wt %). It was extracted with DCM twice. The combined DCM layers were washed with 5 wt % brine, dried over Na₂SO₄, and concentrated. The residue was co-evaporated with toluene three times before it was re-dissolved in DCM (22.06 mL, 342.873 mmol), into which was then added 4-(hydroxymethyl)phenyl pivalate (1.548 g, 7.430 mmol), DBU (0.799 mL, 5.301 mmol) and N-methylpyrrolidine (0.551 ml, 5.301 mmol) at 0° C. After addition, the ice bath was removed and the reaction mixture was stirred at room temperature overnight. It was then cooled in ice bath and quenched with sodium dihydrogen phosphate aqueous solution (73.2 ml, 53.012 mmol, 10 wt %). It was extracted with DCM twice, and the combined DCM layers were washed with 5 wt % brine, dried over Na₂SO₄, and concentrated. Silica gel column purification of the concentrate with heptane-ethyl acetate afforded 2.08 g of the product.

¹H NMR (400 MHz, CHLOROFORM-d) δ ppm 8.13 (s, 1H), 7.78 (s, 1H), 7.53 (d, J=8.5 Hz, 2H), 7.04 (dd, J=8.5, 2.3 Hz, 2H), 6.39 (dd, J=6.4, 6.4 Hz, 1H), 5.60 (m, 2H), 4.60 (m, 1H), 3.99 (m, 1H), 3.85 (dd, J=11.2, 4.0 Hz, 1H), 3.77 (dd, J=11.2, 3.2 Hz, 1H), 3.22 (br s, 1H), 2.56 (m, 1H), 2.40 (ddd, J=13.0, 6.0, 3.8 Hz, 1H), 1.35 (s, 9H), 1.28 (d, J=6.8 Hz, 6H), 0.91 (s, 9H), 0.91 (s, 9H), 0.10 (s, 6H), 0.08 (s, 6H).

MS (ESI) m/z: calculated for C₃₈H₆₁N₅O₇Si₂ [M+H]⁺: 756.4; found 756.3.

### Synthesis of 4-(((9-((2R,4S,5R)-4-((tert-butyldimethylsilyl)oxy)-5-(hydroxymethyl)tetrahydrofuran-2-yl)-2-isobutyramido-9H-purin-6-yl)oxy)methyl)phenyl pivalate

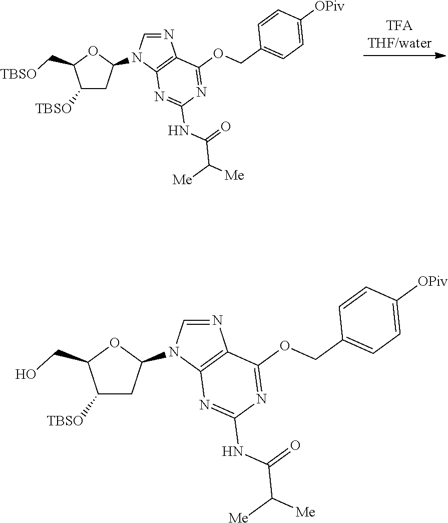

To a solution of 4-(((9-((2R,4S,5R)-4-((tert-butyldimethylsilyl)oxy)-5-(((tert-butyldimethylsilyl)oxy)methyl)tetrahydrofuran-2-yl)-2-isobutyramido-9H-purin-6-yl)oxy)methyl)phenyl pivalate (2.08 g, 2.672 mmol) in THF (22.78 mL) and water (3.80 mL) was added TFA (0.618 ml, 8.016 mmol) dropwise at 0° C. The reaction mixture was stirred with ice bath overnight before it was quenched with sat. sodium bicarbonate aqueous solution (42.1 mL, 40.078 mmol). It was extracted with EtOAc (100 mL 2×), and combined EtOAc layers were washed with half sat. brine, dried over Na₂SO₄, and concentrated. Silica gel column purification of the concentrate with heptane-ethyl acetate afforded 0.83 g of product.

¹H NMR (400 MHz, CHLOROFORM-d) δ ppm 7.87 (s, 1H), 7.76 (s, 1H), 7.55 (d, J=8.4 Hz, 2H), 7.04 (m, J=8.4 Hz, 2H), 6.24 (dd, J=8.8, 6.0 Hz, 1H), 5.55-5.70 (m, 2H), 5.01 (br dd, J=10.0, 2.8 Hz, 1H), 4.78 (br d, J=5.2 Hz, 1H), 4.10 (m, 1H), 3.94 (br d, J=12.4 Hz, 1H), 3.78 (m, 1H), 2.97 (ddd, J=13.2, 8.4, 5.2 Hz, 1H), 2.84 (m, 1H), 2.23 (ddd, J=11.6, 5.6, 1.6 Hz, 1H), 1.35 (s, 9H), 1.28 (d, J=6.8 Hz, 6H), 0.92 (s, 9H), 0.12 (s, 3H), 0.11 (s, 3H).

MS (ESI) m/z: calculated for C₃₂H₄₇N₅O₇Si [M+H]⁺: 642.3; found 642.3.

### Synthesis of 4-(((9-((2R,4S,5R)-4-((tert-butyldimethylsilyl)oxy)-5-(((chloro(dimethylamino)phosphoryl)oxy)methyl)tetrahydrofuran-2-yl)-2-isobutyramido-9H-purin-6-yl)oxy)methyl)phenyl pivalate

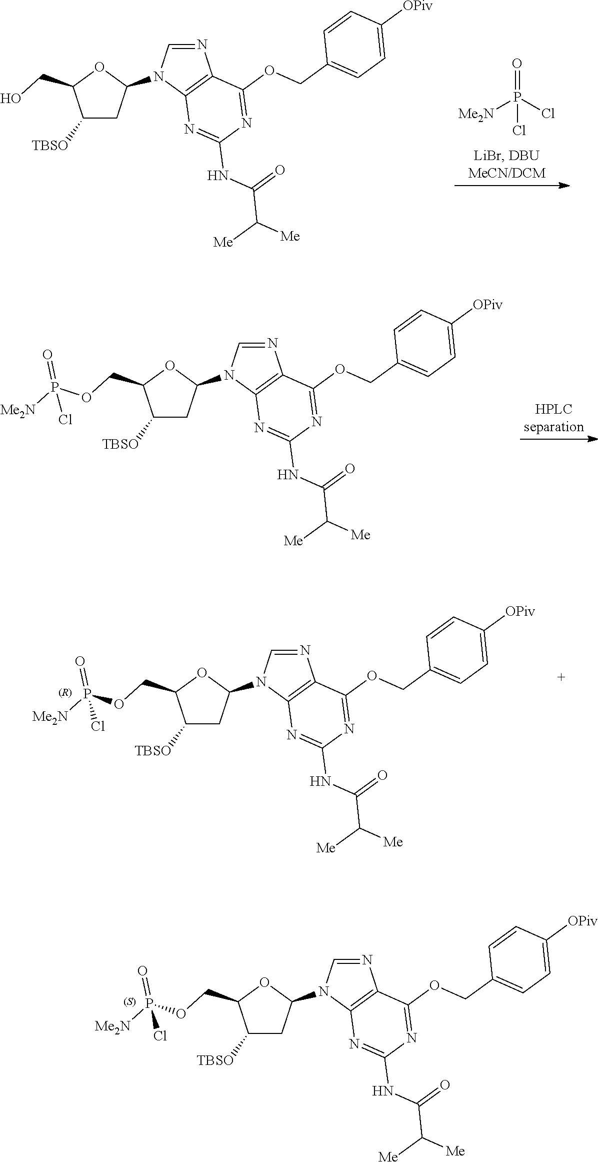

4-(((9-((2R,4S,5R)-4-((tert-butyldimethylsilyl)oxy)-5-(hydroxymethyl)tetrahydrofuran-2-yl)-2-isobutyramido-9H-purin-6-yl)oxy)methyl)phenyl pivalate (0.83 g, 1.293 mmol) was co-evaporated with anhydrous MeCN once before it was dissolved in MeCN (8.46 mL, 162.048 mmol) and DCM (8.46 ml, 131.539 mmol), into which lithium bromide (0.337 g, 3.879 mmol) and DBU (0.585 ml, 3.879 mmol) was added. The mixture was then cooled in an ice bath, into which dimethylphosphoramidic dichloride (0.200 mL, 1.681 mmol) was added. The reaction mixture was stirred in an ice bath for 1 hr before it was quenched with aqueous solution of citric acid (16.40 mL, 8.535 mmol, 10 wt %) at 0° C. It was extracted with DCM (42.3 mL 2×). The combined DCM layers were washed subsequently with water twice and half sat. brine, dried over Na₂SO₄, and concentrated. Silica gel column purification of the concentrate with heptane-ethyl acetate afforded 0.60 g of the product. MS (ESI) m/z: calculated for C₃₄H₅₂ClN₆O₈PSi [M+H]⁺: 767.3; found 767.0.

The stereoisomer mixture was subjected to the following HPLC separation method to isolate the (R) and (S)-stereoisomers of the product:

### HPLC Separation Method

Column: Chiralpak IA, 21×250 mm 5 u
Flowrate: 20 m/min
Mobile Phase: 65% Heptane 35% EA
Gradient: Isocratic
Runtime 25 mins
Injection Volume: 500 uL 40 mg/ml concentration
Detection: 260 nm

### Peak 1, Rt at 11.70 Min

### 4-(((9-((2R,4S,5R)-4-((tert-butyldimethylsilyl)oxy)-5-((((R)-chloro(dimethylamino)phosphoryl)oxy)methyl)tetrahydrofuran-2-yl)-2-isobutyramido-9H-purin-6-yl)oxy)methyl)phenyl pivalate

¹H NMR (400 MHz, CHLOROFORM-d) δ ppm 8.44 (br s, 1H), 7.90 (s, 1H), 7.58 (d, J=8.4 Hz, 2H), 7.03 (d, J=8.4 Hz, 2H), 6.32 (dd, J=6.8, 6.8 Hz, 1H), 5.56-5.72 (m, 2H), 4.80 (m, 1H), 4.73 (m, 1H), 4.15-4.30 (m, 2H), 3.08 (m, 1H), 2.97 (m, 1H), 2.74 (s, 3H), 2.70 (s, 3H), 2.37 (ddd, J=13.2, 6.4, 3.2 Hz, 1H), 1.34 (s, 9H), 1.28 (d, J=6.8 Hz, 6H), 0.92 (s, 9H), 0.13 (s, 3H), 0.12 (s, 3H). ³¹P NMR (162 MHz, CHLOROFORM-d) δ ppm 19.04 (s, 1P).

### Peak 2, Rt at 17.94 Min

### 4-(((9-((2R,4S,5R)-4-((tert-butyldimethylsilyloxy)-5-((((S)-chloro(dimethylamino)phosphoryl)oxy)methyl)tetrahydrofuran-2-yl)-2-isobutyramido-9H-purin-6-yl)oxy)methyl)phenyl pivalate

¹H NMR (400 MHz, CHLOROFORM-d) δ ppm 8.31 (s, 1H), 7.89 (s, 1H), 7.57 (d, J=8.4 Hz, 2H), 7.03 (d, J=8.4 Hz, 2H), 6.32 (dd, J=6.8, 6.8 Hz, 1H), 5.56-5.70 (m, 2H), 4.78 (m, 1H), 4.55 (m, 1H), 4.39 (m, 1H), 4.21 (m, 1H), 3.00 (m, 2H), 2.67 (s, 3H), 2.63 (s, 3H), 2.37 (ddd, J=13.2, 6.4, 3.2 Hz, 1H), 1.34 (s, 9H), 1.28 (d, J=6.8 Hz, 3H), 1.27 (d, J=6.8 Hz, 3H), 0.92 (s, 9H), 0.15 (s, 3H), 0.13 (s, 3H). ³¹P NMR (162 MHz, CHLOROFORM-d) δ ppm 19.13 (s, 1P).

What is claimed is:

1. A bis-protected, activated guanine monomer according to Formula I: 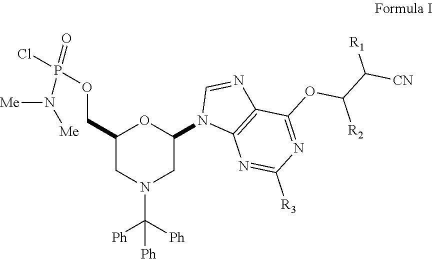
wherein R₁ and R₂ are selected from a H, a halogen, (R)-methyl or (S)-methyl, a C₁-C₄ alkyl, a phenyl, an aryl, a cycloalkyl or any combination thereof, or wherein R₁ and R₂ together form a C₃-C₈ cycloalkyl that is saturated or unsaturated, and that is unsubstituted or substituted with one or more C₁-C₆ alkyl; and
wherein R₃ is selected from NH₂, —NHC(O)R₇, —NHC(O)OR₇, 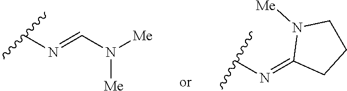 [SMILES: */N=C/N(C)C ; */N=C1/CCCN1C]
and where R₇ may be a C₁-C₆ alkyl, isopropyl, 2,2,2-trichloroethyl, benzyl or aryl.

2. The bis-protected, activated guanine monomer according to claim 1, wherein the guanine monomer is a stereoisomer of Formula I comprising a structure according to Formula Ia:

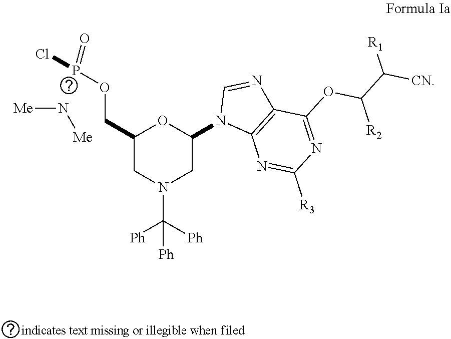

3. The bis-protected, activated guanine monomer according to claim 1, wherein the guanine monomer is a stereoisomer of Formula I comprising a structure according to Formula Ib:

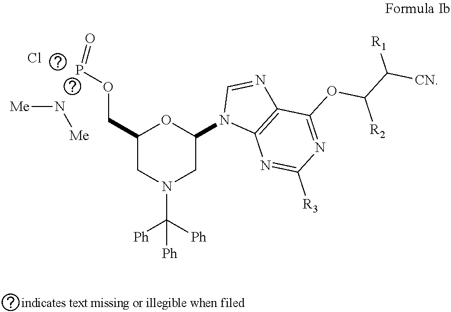

4. The bis-protected, activated guanine monomer according to claim 1, wherein R t is H and R₂ is (R)-methyl or (S)-methyl.

5. The bis-protected, activated guanine monomer according to claim 1, wherein R₁ is (R)-methyl or (S)-methyl and R₂ is H.

6. A bis-protected, activated guanine monomer according to Formula II: 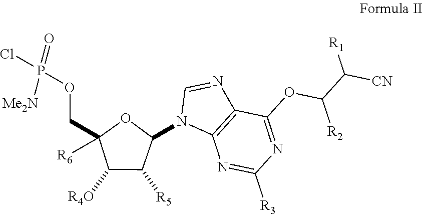
wherein R₁ and R₂ are selected from a H, a halogen, (R)-methyl or (S)-methyl, a C₁-C₄ alkyl, a phenyl, an aryl, a cycloalkyl or any combination thereof, or wherein R₁ and R₂ together form a C₃-C₇ cycloalkyl ring or heterocycle ring comprising a nitrogen or oxygen that is either unsaturated or saturated and that is either unsubstituted or substituted with C₁-C₆ alkyl;
wherein R₃ is selected from NH₂, —NHC(O)R₇, —NHC(O)OR₇, 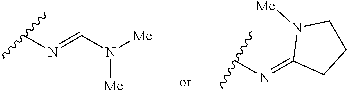 [SMILES: */N=C/N(C)C ; */N=C1/CCCN1C]
and where R₇ may be a C₁-C₆ alkyl, isopropyl, 2,2,2-trichloroethyl, benzyl or aryl;
wherein R₄ is selected from a H, trityl (Tr), monomethoxytrityl (MMTr), dimethoxytrityl (DMTr), —Si(R₆)₃, wherein R₆ is a C₁-C₆ alkyl or an aryl; and
wherein R₅ is selected from H, —OMe, —F or —OCH₂CH₂OMe, or wherein R₅ and R₆ can be linked together to form a C₃ to C₇ cycloalkyl ring or heterocycle ring comprising oxygen and/or nitrogen, all of which may be saturated or unsaturated, and may be unsubstituted or substituted with C₁-C₆ alkyl.

7. The bis-protected, activated guanine monomer according to claim 6, wherein the guanine monomer is a stereoisomer of Formula II comprising a structure according to Formula IIa:

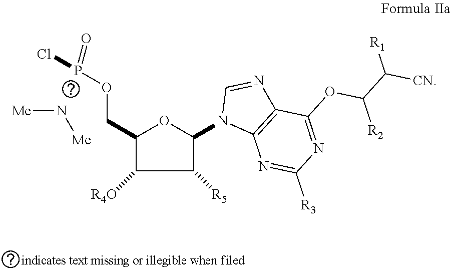

8. The bis-protected, activated guanine monomer according to claim 6, wherein the guanine monomer is a stereoisomer of Formula II comprising a structure according to Formula IIb:

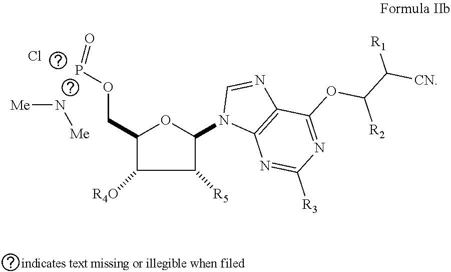

9. The bis-protected, activated guanine monomer according to claim 6, wherein R₁ is H and R₂ is (R)-methyl or (S)-methyl.

10. The bis-protected, activated guanine monomer according to claim 6, wherein R₁ is (R)-methyl or (S)-methyl and R₂ is H.

11. A method for producing a bis-protected, activated guanine monomer according to claim 1, wherein the method comprises:
i.) reacting a protected guanine monomer according to Formula (III): 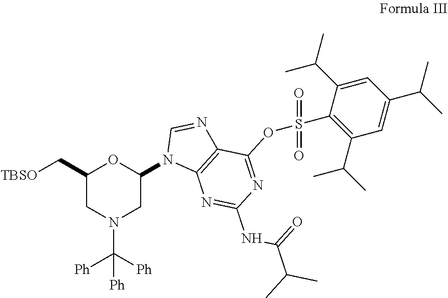 [SMILES: CC(C)C(=O)Nc1nc(OS(=O)(=O)c2c(C(C)C)cc(C(C)C)cc2C(C)C)c2ncn([C@H]3CN(C(c4ccccc4)(c4ccccc4)c4ccccc4)C[C@@H](CO[Si](C)(C)C(C)(C)C)O3)c2n1]
with an alcohol in the presence of a base and an activating agent to produce a protected guanine intermediate according to Formula IV: 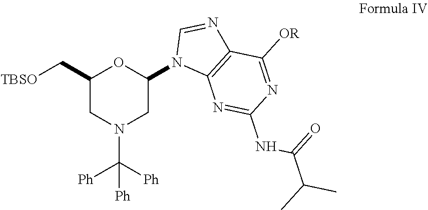
wherein R is selected from the group consisting of the following structures: 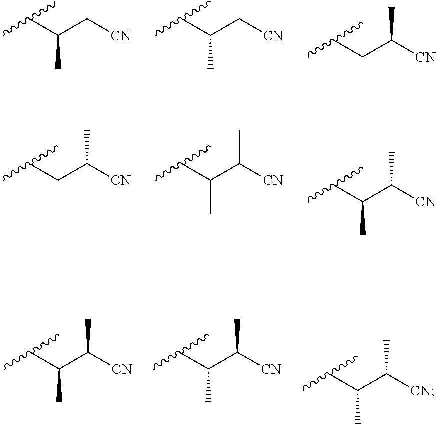
ii.) reacting the protected guanine intermediate according to Formula IV with triethylamine trihydrofluoride to produce a deprotected guanine intermediate according to Formula V: 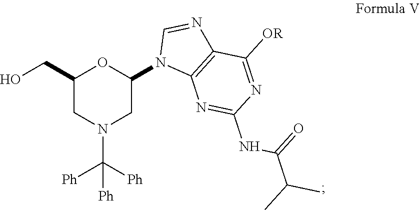
iii.) reacting the deprotected guanine intermediate according to Formula V with lithium bromide, a second activating agent and N,N-dimethylphosphoramic dichloride to create the bis-protected, activated guanine monomer.

12. The method according to claim 11, wherein the first and second activating agent are DBU and the base is N-methylpyrrolidine.

13. A method of creating a bis-protected, activated guanine monomer, wherein the method comprises:
i) reacting a guanine monomer according to Formula VI: 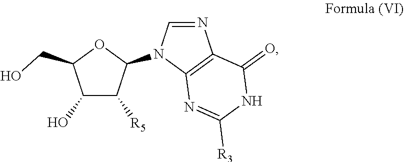
with a first protecting agent to produce a first protected guanine monomer according to Formula (VII): 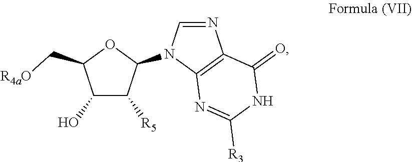
wherein R₃ is selected from NH₂, —NHC(O)R₇ or —NHC(O)OR₇, where R₇ is C₁-C₆ alkyl;
isopropyl, benzyl, 2,2,2-trichloroethyl, 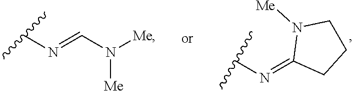 [SMILES: */N=C/N(C)C ; */N=C1/CCCN1C]
or aryl;
wherein R_{4A} is selected from trityl (Tr), monomethoxytrityl (MMTr), dimethoxytrityl (DMTr), or —Si(R₆)₃, where R₆ is C₁-C₆ alkyl or aryl;
wherein R₅ is selected from H, —OMe, —OMOE-F or —OCH₂CH₂OMe;
ii) reacting the protected guanine monomer of Formula (VII) with a second protecting agent to produce a protected guanine monomer according to Formula (VIII): 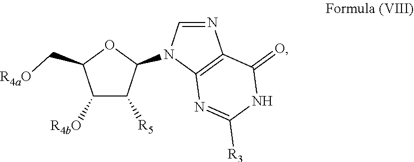
wherein R_{4B} is selected from trityl (Tr), monomethoxytrityl (MMTr), dimethoxytrityl (DMTr), or —Si(R₆)₃, where R₆ is C₁-C₆ alkyl or aryl;
iii) reacting the second protected guanine monomer of Formula (VIII) with an activating agent to produce a protected guanine monomer according to Formula (IX): 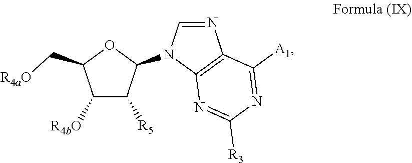
wherein A₁ is a leaving group formed from the reaction with the activating agent;
iv) reacting the protected guanine monomer of Formula (IX) with an alcohol to produce a protected guanine monomer according to Formula (X): 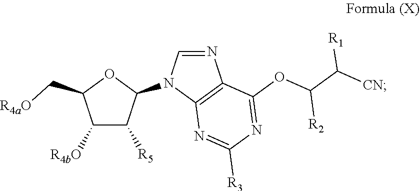
v) deprotecting the protected guanine monomer according to Formula (X) with a deprotecting agent to produce a protected guanine monomer according to Formula (XI): 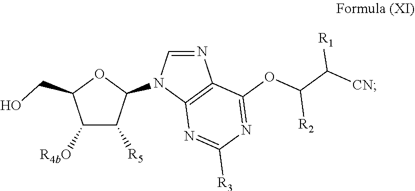
vi) reacting the protected guanine monomer according to Formula (XI) with an electrophile to produce a protected guanine monomer according to Formula II

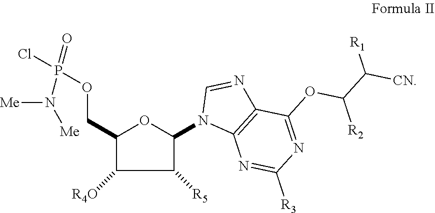

14. The method according to claim 13, wherein the second protecting agent is tert-Butyldimethylsilyl chloride.

15. The method according to claim 13, wherein the activating agent is 2,4,6-Triisopropylbenzenesulfonyl chloride.

16. The method according to claim 13, wherein A₁ is

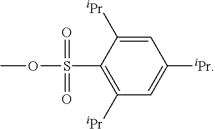 [SMILES: COS(=O)(=O)c1c(C(C)C)cc(C(C)C)cc1C(C)C]

17. The method according to claim 13, wherein the third protecting agent is

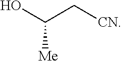

18. The method according to claim 13, wherein the deprotecting agent is trifluoroacetic acid.

19. The method according to claim 13, wherein the electrophile is

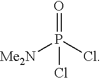

20. A method of creating a bis-protected, activated guanine monomer, wherein the method comprises:
i.) reacting the guanine monomer of Formula (IX) 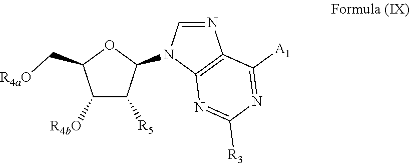
wherein R₃ is —NHC(O)R₇, where R₇ is isopropyl;
wherein R_{4A} and R_{4B} are selected from trityl (Tr), monomethoxytrityl (MMTr),
dimethoxytrityl (DMTr), or —Si(R₆)₃, where R₆ is C₁-C₆ alkyl or aryl;
wherein R₅ is selected from H, —OMe, —OMOE-F or —OCH₂CH₂OMe;
wherein A₁ is a leaving group;
with 4-(hydroxymethyl)phenyl pivalate to produce a protected guanine monomer according to Formula (XII): 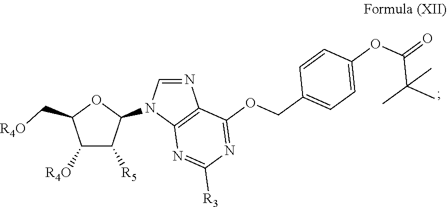
ii.) deprotecting the protected guanine monomer according to Formula (XII) with a deprotecting agent to produce a protected guanine monomer according to Formula (XIII): 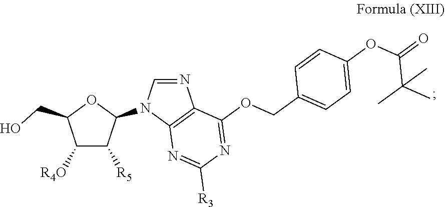
iii.) reacting the protected guanine monomer according to Formula (XIII) with an electrophile to produce a compound with the following structure:

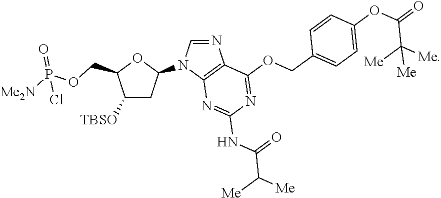 [SMILES: CC(C)C(=O)Nc1nc(OCc2ccc(OC(=O)C(C)(C)C)cc2)c2ncn([C@H]3C[C@H](O[Si](C)(C)C(C)(C)C)[C@@H](COP(=O)(Cl)N(C)C)O3)c2n1]

21. The method according to claim 20, wherein the protecting agent is 4-(hydroxymethyl)phenyl pivalate.

22. A bis-protected, activated guanine monomer according to any one of the following structures: 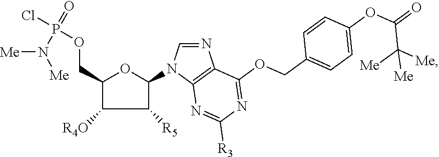 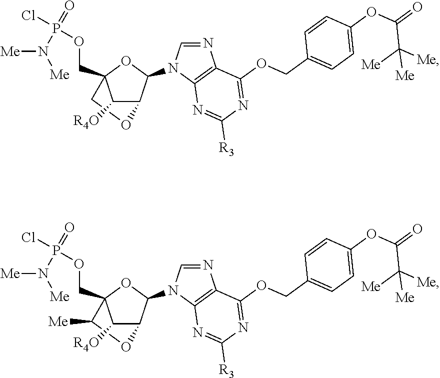
wherein R₃ is selected from NH₂, —NHC(O)R₇, —NHC(O)OR₇, 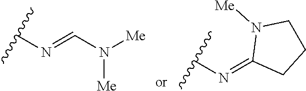 [SMILES: */N=C/N(C)C ; */N=C1/CCCN1C]
and where R₇ may be a C₁-C₆ alkyl, isopropyl, 2,2,2-trichloroethyl, benzyl or aryl;
wherein R₄ is selected from a H, trityl (Tr), monomethoxytrityl (MMTr), dimethoxytrityl (DMTr), —Si(R₆)₃, wherein R₆ is a C₁-C₆ alkyl or an aryl; and
wherein R₅ is selected from H, —OMe, —F or —OCH₂CH₂OMe.
